(19) United States
(12) Patent Application Publication
(10) Pub. No.: US 2025/0107893 A1
(43) Pub. Date: Apr. 3, 2025

(54) ANCHOR DELIVERY AND ASSESSMENT

(71) Applicant: Edwards Lifesciences Innovation (Israel) Ltd., Caesarea (IL)

(72) Inventors: Andrew Charles May, Irvine, CA (US); Nicolas Schleiger, Huntington Beach, CA (US)

(21) Appl. No.: 18/981,498

(22) Filed: Dec. 14, 2024

Related U.S. Application Data
(63) Continuation of application No. PCT/IB2023/056307, filed on Jun. 19, 2023.
(60) Provisional application No. 63/489,147, filed on Mar. 8, 2023.
(60) Provisional application No. 63/415,595, filed on Oct. 12, 2022.
(60) Provisional application No. 63/366,713, filed on Jun. 21, 2022.

(51) Int. Cl. A61F 2/24

(52) U.S. Cl. CPC A61F 2/2466; A61F 2/2445; A61F 2220/0016

(57) ABSTRACT

A method involves delivering an elongate shaft to a target tissue location, the elongate shaft comprising a flex portion configured to move between a first state and a second state, moving the flex portion from the first state to the second state to facilitate driving a first tissue anchor into the target tissue location, and moving the flex portion from the second state to the first state while maintaining contact between the flex portion and the first tissue anchor.

### RELATED APPLICATIONS

This application is a continuation of International Patent Application No. PCT/IB2023/056307, filed Jun. 19, 2023 and entitled ANCHOR DELIVERY AND ASSESSMENT SYSTEMS, which claims priority to U.S. Provisional Patent Application Ser. No. 63/366,713, filed on Jun. 21, 2022 and entitled ANCHOR DRIVERS, U.S. Provisional Patent Application Ser. No. 63/415,595, filed on Oct. 12, 2022 and entitled ANCHOR DELIVERY SYSTEMS, and U.S. Provisional Patent Application Ser. No. 63/489,147, filed on Mar. 8, 2023 and entitled ANCHOR DELIVERY AND ASSESSMENT SYSTEMS, the complete disclosures of which are hereby incorporated by reference in their entirety.

### BACKGROUND

Annuloplasty generally involves the remodeling of tissue of a native heart valve annulus. Annulus remodeling can be performed by pulling tissue about the annulus to a new shape. Tensioning wires/lines connecting tissue anchors and/or other implant devices can be used to facilitate medical procedures, such as annuloplasty or other remodeling procedures.

### SUMMARY

Described herein are one or more methods and/or devices for delivering, driving, and/or assessing placement and/or anchoring of one or more tissue anchors.

Some implementations of the present disclosure relate to a system for assessing anchoring of one or more tissue anchors, the system including: an outer shaft including an inner lumen; and an inner shaft configured to fit at least partially within the inner lumen of the outer shaft and drive one or more tissue anchors, wherein the inner shaft has a generally flexible structure relative to the outer shaft.

In some implementations, the techniques described herein relate to a system, wherein the inner shaft includes one or more cavities configured to improve flexibility of the inner shaft.

In some implementations, the techniques described herein relate to a system, wherein the outer shaft is configured to advance and retract independently of the inner shaft.

In accordance with some implementations of the present disclosure, a method for assessing anchoring of one or more tissue anchors includes delivering an outer shaft and an inner shaft to a target tissue location, the outer shaft at least partially enclosing the inner shaft.

In some implementations, the method further includes attaching the inner shaft to a tissue anchor.

In some implementations, the method further includes retracting the outer shaft to expose a distal portion of the inner shaft.

In some implementations, the method further includes advancing the inner shaft to create slack in the distal portion of the inner shaft.

In some implementations, the method further includes assessing the tissue anchor with the inner shaft attached to the tissue anchor.

In some implementations, the method further includes advancing the outer shaft to drive the tissue anchor into tissue.

In some implementations, the inner shaft is generally flexible relative to the outer shaft. In some implementations, the inner shaft includes one or more cavities configured to improve flexibility of the inner shaft.

In some implementations, the outer shaft is configured to advance and retract independently of the inner shaft.

In some implementations, the method further includes, in response to determining that the tissue anchor is suitably anchored, removing the inner shaft from the tissue anchor.

In some implementations, advancing the inner shaft to create slack in the distal portion of the inner shaft involves allowing the distal portion of the inner shaft to bend in multiple directions.

In some implementations, the method further includes detaching the inner shaft from the tissue anchor.

In some implementations, the method further includes attaching the inner shaft to a second tissue anchor. In some implementations, the method further includes retracting the outer shaft to expose a distal portion of the inner shaft.

In some implementations, the method further includes advancing the inner shaft to create slack in the distal portion of the inner shaft.

In some implementations, the method further includes assessing the second tissue anchor with the inner shaft attached to the second tissue anchor.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations of the present disclosure, a method for delivering one or more tissue anchors comprises delivering an elongate shaft carrying one or more tissue anchors at a distal end of the elongate shaft to a target tissue location.

In some implementations, the elongate shaft comprises a flex portion configured to move between a first state and a second state.

In some implementations, the method can further comprise moving the flex portion from the first state to the second state to facilitate driving the one or more tissue anchors into the target tissue location and moving the flex portion from the second state to the first state.

In some implementations, moving the flex portion from the first state to the second state can involve compressing the flex portion.

In some implementations, moving the flex portion from the first state to the second state involves twisting the elongate shaft until a gap forms in the flex portion.

In some implementations, the flex portion comprises at least one detachable tab between segments of the flex portion. The segments can be ring-shaped.

In some implementations, the flex portion can comprise a network of interlocking teeth. In some implementations, the flex portion comprises a coil extending through a lumen of the flex portion.

In some implementations, the coil is configured to interconnect segments of the flex portion. In some implementations, the flex portion can comprise a cut through the elongate shaft.

In some implementations, moving the flex portion from the first state to the second state involves pulling one or more pull wires extending at least partially through the flex portion.

In some implementations, the flex portion includes two or more segments. In some implementations, the one or more pull wires interconnect the two or more segments.

In some implementations, moving the flex portion from the second state to the first state involves releasing the one or more pull wires.

In some implementations, in response to determining that the first tissue anchor is not suitably anchored, the method includes moving the flex portion from the first state to the second state to facilitate further driving the first tissue anchor into the target tissue location.

In some implementations, in response to determining that the first tissue anchor is suitably anchored, the method includes removing the flex portion from the first tissue anchor.

In some implementations, the method further comprises moving the first tissue anchor from the first state to the second state to facilitate driving a second tissue anchor into a second target tissue location.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In some implementations, a system for delivering one or more tissue anchors comprises an elongate shaft carrying one or more tissue anchors at a distal end of the elongate shaft to a target tissue location. In some implementations, the elongate shaft comprises a flex portion configured to move between a first state and a second state.

In some implementations, the system further comprises an inner shaft configured to extend at least partially through a lumen of the elongate shaft. In some implementations, the system can further comprise an outer shaft configured to at least partially enclose the elongate shaft.

In some implementations, the flex portion can be configured to move from the first state to the second state by compressing the flex portion.

In some implementations, the flex portion is configured to form a gap in response to twisting of the elongate shaft.

In some implementations, the flex portion comprises at least one detachable tab between segments of the flex portion. The segments can be ring-shaped.

In some implementations, the flex portion can comprise a network of interlocking teeth. In some implementations, the flex portion comprises a coil extending through a lumen of the flex portion.

In some implementations, the coil is configured to interconnect segments of the flex portion. The flex portion can comprise a cut through the elongate shaft.

In some implementations, the system can further comprise one or more pull wires configured to extend at least partially through the flex portion. In some implementations, the one or more pull wires extend at least partially through multiple segments of the flex portion.

Any of the various systems, assemblies, devices, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, apparatus, etc. (e.g., with heat, radiation, ethylene oxide, hydrogen peroxide, etc.).

For purposes of summarizing the disclosure, certain aspects, advantages, and novel features have been described. It is to be understood that not necessarily all such advantages can be achieved in accordance with any particular implementation. Thus, the disclosed implementations can be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica or overlaid on one or more of the physical components. Virtual elements can be presented on any combination of screens, headsets, holographically, projected, loudspeakers, headphones, pressure transducers, temperature transducers, or using any combination of suitable technologies.

### BRIEF DESCRIPTION OF THE DRAWINGS

Various implementations are depicted in the accompanying drawings for illustrative purposes and should in no way be interpreted as limiting the scope of the inventions. In addition, various features of different disclosed implementations can be combined to form additional implementations, which are part of this disclosure. Throughout the drawings, reference numbers may be reused to indicate correspondence between reference elements.

FIG. 1 illustrates an example representation of a heart and associated anatomy having various features relevant to certain implementations of the present inventive disclosure.

FIG. 2 shows an overhead view of the annuloplasty implant system implanted in a native heart valve annulus in accordance with one or more implementations.

FIG. 3 illustrates an example implant system in which one or more tissue anchors anchor an annuloplasty tube to annuloplasty tissue of a mitral valve 3, in accordance with one or more implementations.

FIG. 4 illustrates an example tissue anchor in accordance with one or more implementations.

FIG. 5 illustrates an example delivery system for delivering one or more tissue anchors to a target tissue site in accordance with one or more implementations.

FIG. 6 illustrates a system for delivering one or more tissue anchors into tissue, in accordance with one or more implementations.

FIG. 7 illustrates anchoring of one or more tissue anchors to tissue in accordance with one or more implementations.

FIG. 8 illustrates delivery of one or more tissue anchors into tissue 10 in accordance with one or more implementations.

FIG. 9 illustrates delivery of a delivery shaft into a chamber (e.g., a right atrium) of a heart in accordance with one or more implementations.

FIG. 10 illustrates an example flexible delivery shaft configured for delivery of one or more anchors at various portions of anatomy, which can include at or around a tricuspid valve, mitral valve, and/or other valve.

FIG. 11 illustrates an example assembly for driving one or more anchors into target tissue, in accordance with one or more implementations.

FIG. 12 illustrates at least a portion of an assembly for delivering and/or driving one or more tissue anchors to target tissue locations, in accordance with one or more implementations.

FIG. 13 illustrates an example assembly configured for delivery and/or driving of various tissue anchors to target tissue locations, in accordance with one or more implementations.

FIGS. 14A-14C illustrate an example flex portion for one or more shafts of a tissue anchor delivery assembly in accordance with one or more implementations described herein.

FIG. 15 illustrates an example flex portion comprising one or more rings and/or one or more springs joining the one or more rings, in accordance with one or more implementations.

FIG. 16 illustrates an example flex portion comprising one or more rings and/or one or more cords and/or networks of cords joining the one or more rings, in accordance with one or more implementations.

FIG. 17 illustrates at least a portion of an assembly comprising an example flex portion comprising one or more rings and/or one or more springs joining the one or more rings, in accordance with one or more implementations.

FIG. 18 illustrates at least a portion of an assembly comprising an example flex portion comprising one or more segments including a first segment, a second segment, and/or a third segment, in accordance with one or more implementations.

FIG. 19 illustrates an example flex portion comprising one or more interlocking teeth in accordance with one or more implementations.

FIG. 20 (FIGS. 20-1 and 20-2) is a flowchart illustrating steps of an example process for delivering one or more tissue anchors via a shaft comprising a flex portion in accordance with one or more implementations.

FIG. 21 (FIGS. 21-1 and 21-2) provides example images corresponding to steps of the process of FIG. 20.

FIGS. 22A and 22B illustrate at least a portion of an example delivery shaft comprising a flex portion including one or more springs extending across a break in the delivery shaft, in accordance with one or more implementations.

FIGS. 23A and 23B illustrate driving process for one or more tissue anchors into tissue in accordance with one or more implementations.

FIG. 24 illustrates an example shaft comprising a flex portion in accordance with one or more implementations.

FIGS. 25A and 25B illustrate an example delivery device for delivering one or more catheters and/or medical devices to a target location within a body, in accordance with one or more implementations.

FIG. 26 illustrates an example flex portion comprising one or more pull wires configured to control positioning of one or more rings and/or one or more springs joining the one or more rings, in accordance with one or more implementations.

FIGS. 27A-27C illustrate an example compressible and/or adjustable anchor delivery system configured for delivery of one or more tissue anchors in accordance with one or more examples.

FIGS. 28A-28D illustrate an example flex portion of a delivery catheter in accordance with one or more implementations.

FIG. 29 (FIGS. 29-1 and 29-2) provides a flowchart illustrating an example process for driving one or more tissue anchors and/or for assessing anchoring of one or more tissue anchors in accordance with one or more examples.

FIG. 30 (FIGS. 30-1 and 30-2) provide images associated with steps of the process of FIG. 29.

FIG. 31 (FIGS. 31-1 and 31-2) provides a flowchart illustrating an example method or process for driving one or more tissue anchors and/or for assessing anchoring of one or more tissue anchors in accordance with one or more examples.

FIG. 32 (FIGS. 30-1 and 30-2) provides images associated with steps of the process of FIG. 31.

### DETAILED DESCRIPTION

The headings provided herein are for convenience only and do not necessarily affect the scope or meaning of the claimed invention.

Although certain preferred implementations and examples are disclosed below, inventive subject matter extends beyond the specifically disclosed implementations to other alternative implementations and/or uses and to modifications and equivalents thereof. Thus, the scope of the claims that may arise herefrom is not limited by any of the particular implementations described below. For example, in any method or process disclosed herein, the acts or operations of the method or process can be performed in any suitable sequence and are not necessarily limited to any particular disclosed sequence. Various operations can be described as multiple discrete operations in turn, in a manner that may be helpful in understanding certain implementations; however, the order of description should not be construed to imply that these operations are order dependent. Further, the treatment techniques, methods, operations, steps, etc. described or suggested herein or in the references incorporated herein can be performed on a living subject (e.g., human, other animal, etc.) or on a non-living simulation, such as a cadaver, cadaver heart, simulator, imaginary person, etc.). When performed on a simulation, the body parts, e.g., heart, tissue, valve, etc., can optionally be referred to as “simulated” (e.g., simulated heart, simulated tissue, simulated valve, etc.) and can comprise, for example, computerized and/or physical representations of body parts, tissue, etc.

Additionally, the structures, systems, and/or devices described herein can be embodied as integrated components or as separate components. For purposes of comparing various implementations, certain aspects and advantages of these implementations are described. Not necessarily all such aspects or advantages are achieved by any particular implementation. Thus, for example, various implementations can be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the implementations disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard anatomical terms of location are used herein to refer to the anatomy of animals, and namely humans, with respect to the preferred implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

Implementations described herein relate to devices and/or methods for identifying and/or facilitating successful anchoring of one or more tissue anchors within a patient's body. Various devices used for delivering implants (e.g., tissue anchors) to a heart and/or other anatomy can be required to press hard against structures within the heart and/or on the implant itself. Moreover, the delivery pathways (e.g., the femoral vein) used to deliver devices and/or systems through to reach the heart can include sharp trajectories to, for example, the tricuspid and/or mitral annulus. As a result, devices (e.g., shafts and/or catheters), and particularly distal portions of the devices, may be required to be flexible and/or bendable to allow for delivery through such trajectories. These devices must also be able to translate a perpendicular pressure to cinch cables and/or to position and/or secure anchors delivered via the devices. Implementation devices can comprise various features configured to facilitate navigation to a target location and/or driving of one or more tissue anchors at the target location.

In some implementations, an anchor driver comprises a flex portion and/or an at least partially separable shaft. The shaft can comprise two or more collinear portions, including a distal portion and a proximal portion. In some implementations, the distal portion and the proximal portion may be at least partially detached and/or discontinuous. The anchor driver can comprise one or more connectors configured to at least partially form an attachment between the distal portion and the proximal portion. In some implementations, a connector can comprise a wire and/or coil extending from the distal portion to the proximal portion. The one or more connectors can extend at least partially into the distal portion and/or proximal portion and/or can be configured to extend across a gap and/or break between the distal portion and the proximal portion. In some implementations, the one or more connectors can be configured to maintain contact between the distal portion and the proximal portion via tension and/or mechanical functions. The one or more connectors can be configured to allow for separation between the distal portion and the proximal portion in response to increased resistance at the distal portion and/or proximal portion. For example, separation of the distal portion and the proximal portion may occur when sufficient torque resistance at the distal portion and/or proximal portion is experienced and/or is identified fluoroscopically and/or electrically.

A shaft of an anchor driver can comprise an at least partially disconnected distal portion and proximal portion, which can be at least partially collinear with each other in a default and/or resting state. A spring-loaded wire can hold the two parts together via tension, with the faces of the two parts typically flush with each other at a resting and/or first state. The line of the split can be oblique and/or helical. When sufficient torque resistance is experienced (e.g., because the anchor head has reached the tissue and/or anchoring is complete), continued rotation of the proximal part can cause some separation of the proximal part from the distal part. The wire and/or coil can maintain integrity of the driver, but the resulting gap and/or offset can be detectable fluoroscopically and/or electrically. The spring constant of the spring-loaded wire and/or the characteristics (e.g., size, surface, angle) of the faces of the two parts of the shaft may be known and/or can be used to tune the driver to split at a particular torque resistance.

Some implementations relate to devices configured to be transformable between a flexible state and a generally rigid state. Methods described herein provide for delivering a shaft in a flexible form and modifying the shaft to a generally rigid bar and/or shaft configured for driving one or more anchors.

Shafts and/or catheters described herein can be used for delivery of anchors configured to ride on a wire and/or line. In some implementations, the wire can be cinched as a desired number of anchors are placed to determine an appropriate point to end the procedure. It can be difficult for anchor drives to allow for applied tension on the wire (e.g., for an anchoring check) due to the flexibility of some anchor drives. Implementations described herein can advantageously resist tension associated with cinching to allow for cinching at one or more points and/or for securing anchors at points along the wire. In some implementations, a delivery system comprises a compressible drive shaft that can be compressed from a flexible state to compressed state using a rotatable nut and/or one or more pull wires. In some implementations, a shaft can be at least partially composed of Nitinol and/or other shape memory alloys.

The present disclosure relates to anchoring devices, tensioning devices, systems, and methods for anchoring and/or adjusting tension in a wire or other line, which can be coupled to one or more tissue anchors or other implant device(s). Such implementations can involve linear/axial actuation of one or more anchors and/or tensioned lines. The term “line” is used herein according to its broad and ordinary meaning and may refer to any elongate wire, tether, cord, strip, strand, suture, rope, filament, tie, string, ribbon, strap, or portion thereof, or other type/form of material used in medical procedures to tether, cinch, secure, align, tie, hold, or otherwise control/manipulate implant devices or components (e.g., tissue anchors). Furthermore, implementations of the present disclosure can be implemented in connection with non-surgical and/or non-biological line/wire tensioning. Furthermore, in some contexts herein, the terms “tether,” “wire,” and “line” can be used substantially interchangeably. In addition, use of the singular form of any of the line-related terms listed above, including the terms “tether” and “wire,” may be used to refer to a single line/cord, or to a portion thereof.

In some implementations, the present disclosure relates to systems, devices, and methods for driving and/or anchoring one or more tissue anchors associated with an implant device/assembly. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

Certain implementations are disclosed herein in the context of cardiac implant devices. However, although certain principles disclosed herein are particularly applicable to the anatomy of the heart, it should be understood that tissue-anchor-based implant devices in accordance with the present disclosure can be implanted in, or configured for implantation in, any suitable or desirable anatomy.

The anatomy of the heart is described below to assist in the understanding of certain inventive concepts disclosed herein. In humans and other vertebrate animals, the heart generally comprises a muscular organ having four pumping chambers, wherein the blood flow therein is at least partially controlled by various heart valves, namely, the aortic, mitral (or bicuspid), tricuspid, and pulmonary valves. The valves can be configured to open and close in response to pressure gradients present during various stages of the cardiac cycle (e.g., relaxation and contraction) to control the flow of blood to respective regions of the heart and/or to blood vessels (e.g., pulmonary, aorta, etc.). The contraction of the various heart muscles may be prompted by signals generated by the electrical system of the heart, which is discussed in detail below.

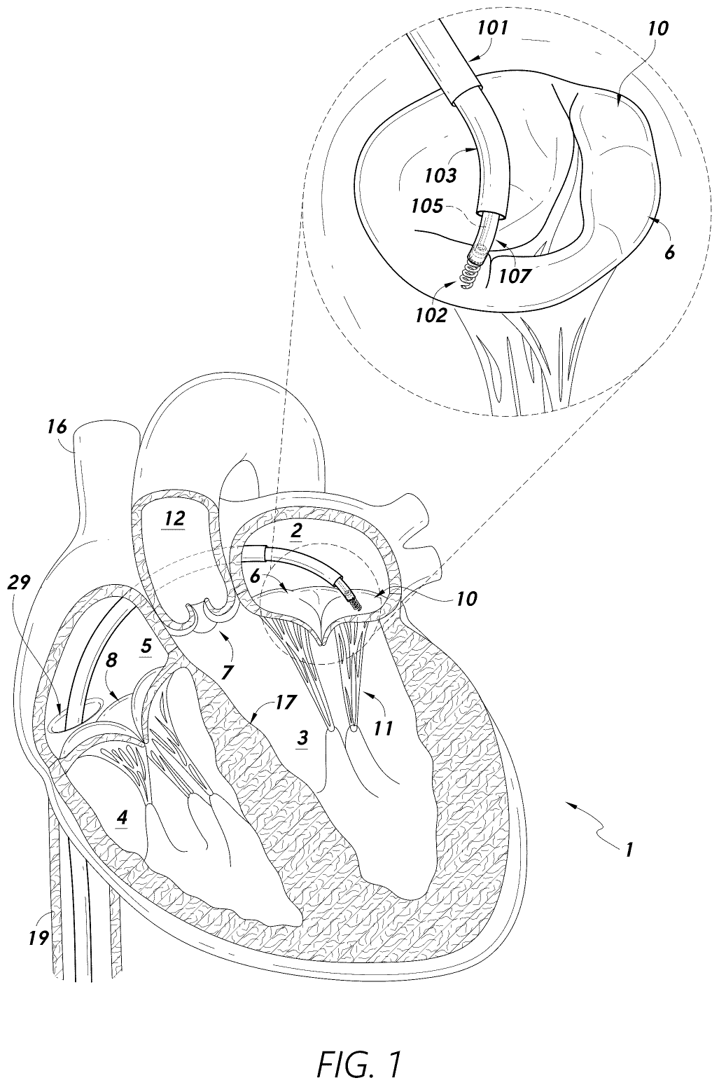

FIG. 1 illustrates an example representation of a heart 1 and associated anatomy having various features relevant to certain implementations of the present inventive disclosure. Generally, the heart 1 includes four chambers, namely the left atrium 2, the left ventricle 3, the right ventricle 4, and the right atrium 5. In terms of blood flow, blood generally flows from the right ventricle 4 into the pulmonary artery via the pulmonary valve (not shown for clarity), which separates the right ventricle 4 from the pulmonary artery and is configured to open during systole so that blood may be pumped toward the lungs and close during diastole to prevent blood from leaking back into the heart from the pulmonary artery. The pulmonary artery carries deoxygenated blood from the right side of the heart to the lungs.

In addition to the pulmonary valve, the heart 1 further includes the tricuspid valve 8, the aortic valve 7, and the mitral valve 6. The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally have associated therewith a relatively dense fibrous/collagenous ring-type structure, referred to herein as the valve annulus, as well as a plurality of leaflets or cusps attached to the annulus. Generally, the size of the leaflets or cusps can be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

With respect to heart valves and associated interventions (e.g., annuloplasty procedures), particular reference is made herein to the mitral valve 6. However, it should be understood that any description herein of anatomy and/or devices or procedures associated with the mitral valve can apply to the other valves of the heart (e.g., tricuspid, aortic); references to the mitral valve specifically are for conveniences and/or due to particular relevance thereof.

With reference to the mitral valve 6, the annulus 10 thereof attaches the mitral leaflets 13 and the left atrium 2 to the ostium of the left ventricle 3 and the aortic root. Under normal conditions, the mitral valve 6 undergoes significant dynamic changes in shape and size throughout the cardiac cycle. These changes are primarily due to the dynamic motion of the surrounding mitral valve annulus 10. Throughout the cardiac cycle, the annulus 10 generally undergoes a sphincter motion, narrowing down the orifice area during systole to facilitate coaptation of the leaflets 13 and widening during diastole to allow for relatively easy diastolic filling of the left ventricle 3. This motion can be further enhanced by a pronounced three-dimensional configuration during systole, which may embody a characteristic saddle shape. The shape and form of the annulus 10 throughout the cardiac cycle can affect proper leaflet coaptation and/or tissue stresses. Disfunction of a heart valve and/or associated leaflets (e.g., pulmonary valve disfunction) can result in valve leakage and/or other health complications.

The atrioventricular (i.e., mitral and tricuspid) heart valves generally are coupled to a collection of chordae tendineae 11 and papillary muscles 9 for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae 11. A wall of muscle 17, referred to as the septum, separates the left 2 and right 5 atria and the left 3 and right 4 ventricles.

Various problems can disrupt blood flow through the valves of the heart. For example, regurgitation, which is also referred to as valve insufficiency or incompetence, occurs when a valve does not close properly and allows blood to leak backward instead of moving in the proper one-way flow. Regurgitation can cause a decrease in the amount of blood that ultimately travels to the body's organs. In order to compensate for regurgitation, the heart may work harder, which in time can cause enlargement/dilation of the heart and reduced cardiac output. In some cases, ischemic heart disease can cause valvular regurgitation. For example, mitral regurgitation can be caused by the combination of ischemic dysfunction of the papillary muscles and left ventricular dilation that can present in ischemic heart disease, with the subsequent displacement of the papillary muscles and the dilatation of the mitral valve annulus. Valve problems can be present at birth or caused by infections, heart attacks, or heart disease or damage.

Certain heart valve diseases/dysfunction can be treated through the implementation of certain annuloplasty treatments, which can help a deformed valve annulus to regain the physiological form and function of a normal, healthy valve (e.g., mitral valve) apparatus. For example, annuloplasty treatments can involve the implantation of a prosthetic annuloplasty device (e.g., ring-shaped device, or “annuloplasty ring”). Annuloplasty treatments can serve to restore/remodel the annular dimensions of a heart valve annulus, which can promote proper leaflet coaptation and/or provide a broader surface of coaptation. Annuloplasty procedures, including annuloplasty ring implantation, can effectively remodel the annulus to restore the size and physiologic shape (e.g., a D- or kidney-type shape) of the annulus and/or decreases the overall circumference of the annulus.

In some cases, an annuloplasty ring/device is constructed from a core material, such as silicone or metal, which provides the desired rigidity; annuloplasty rings can be designed to be relatively flexible, semirigid, or rigid. Some annuloplasty rings are sheathed in a cloth material (e.g., Dacron or polyester), through which anchoring sutures may be placed. Annuloplasty rings can be formed in various sizes, rigidities, and shapes, and can comprise a partial band or complete ring. The size of an annuloplasty ring may be selected/determined based at least in part on the particular dimensions of the target heart valve/annulus (e.g., height of the anterior leaflet, intercommissural distance, intertrigonal distance, etc.).

Annuloplasty ring implantation procedures can be implemented by accessing the heart valve through the chest wall. For example, open-heart surgical access may be utilized to access the heart and the target valve. Alternatively, certain minimally invasive techniques may be implemented wherein one or more relatively smaller cuts in the chest are implemented to access the valve. Transcatheter annuloplasty interventions can be used, wherein an implant device/assembly is advanced to the target valve annulus through the vasculature of the patient, which may be accessed through the femoral vein or other access site/path. Transcatheter annuloplasty procedures can be preferable to surgical and minimally invasive procedures due to decreased risks to the patient; transcatheter annuloplasty procedures in accordance with aspects of the present disclosure can be particularly desirable for the treatment of patients with mitral regurgitation deemed inoperable or at high surgical risk.

The less invasive, percutaneous implantation of annuloplasty devices in accordance with implementations of the present disclosure provides for relatively safer treatment when compared with conventional open-heart surgery. Furthermore, such procedures may allow for relatively earlier indication for treatment to improve the chance of prognostic benefit. Transcatheter annuloplasty devices comprising tissue anchors coupled by tensioning tether/line connectors in accordance with implementations of the present disclosure, may produce results similar to those of surgical annuloplasty rings, such as with respect to the ability to decrease the septolateral dimension and increase leaflet coaptation. An example system that may be utilized in connection with the tensioning solutions presented herein is the Cardioband Mitral Reconstruction System by Edwards Lifesciences, which is a percutaneous surgical-like direct annuloplasty device that can be implanted in a beating heart. Such devices may be implanted on the posterior annulus under fluoroscopic and transoesophageal echocardiographic (TEE) guidance. Following the implantation of tissue anchors associated with the implant device in or near the native valve annulus, the device can be contracted using wire/line tensioning to remodel the annulus and improve mitral regurgitation.

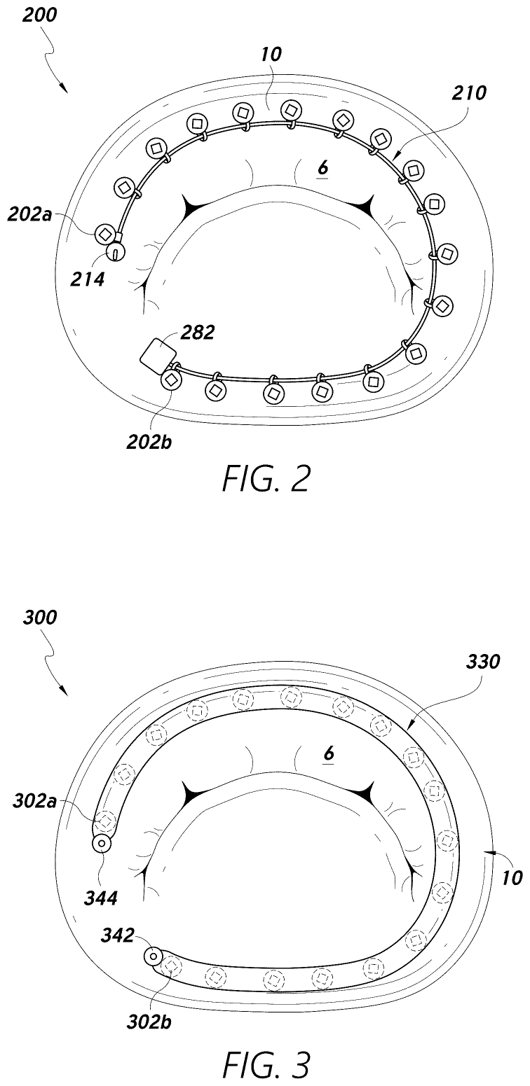

With further reference to FIG. 1, at least a portion of an annuloplasty implant system is shown which can be at least partially adjustable, after implantation of one or more tissue anchors 102. FIG. 2 shows an overhead view of the annuloplasty implant system 200 implanted in a native heart valve annulus 10 in accordance with one or more implementations. In the image of FIG. 2, a tensioning wire 210 is used to interconnect multiple tissue anchors 202 and/or can be tensioned and/or locked. Delivery systems (including, e.g., an outer catheter 101, inner catheter 103, a channel 107, and/or delivery shaft 105, as shown in FIG. 1) can be withdrawn.

The anchors 102 can be secured to the native annulus 10 and/or other anatomy surrounding the valve orifice/leaflets. In some implementations, the implant system can include a sleeve (e.g., polyester) around at least a portion of the line 210 and/or anchors 102/202. Tensioning of the line 210 can effectively shorten the implant system to thereby adjust the implant system size to the patient's needs. Tensioning can be performed under imaging (e.g., echo) guidance as a means of producing and confirming the desired regurgitation reduction.

The tissue anchors 102/202 can be delivered to the target anatomy using a delivery system, which can comprise, for example, one or more delivery and/or guide catheters and/or access sheaths, including an outer catheter 101, an inner catheter 103, a channel 107, and/or a delivery shaft 105 and/or sheath, any of which can be steerable. For mitral repair, at least a portion of the delivery system can be advanced through the interatrial septum. The catheter(s) 101, 103 can include a steerable guide catheter, as well as a flexible tube configured to be advanced through the guide catheter in order to facilitate delivery and implantation of the tissue anchors 102 therefrom. During the delivery, at least a portion of a potentially steerable distal end of such flexible tube can be deployed from the distal end of the guide catheter for advancement to the annulus 10 of the target valve (e.g., mitral valve 6).

The implant system can comprise a plurality of implantable tissue anchors 102/202, which can comprise metal or other biocompatible material. In some implementations, the anchors 1020/202 are corkscrew-type tissue anchors having a proximal drive head, which can be engaged by a drive shaft 105 to embed the anchors in the target tissue. The delivery system can include anchor delivery shaft(s) for advancing the anchors 102/202 from a distal end of the delivery system. In some implementations, the tissue anchors 102/202 comprise stainless steel anchors that are between 5-10 mm in length (e.g., approximately 6 mm). The anchors 102/202 can be used to fasten the connecting line/wire 210 to the target tissue (e.g., annulus 10). Although eighteen anchors 202 are shown in FIG. 2, it should be understood that any number of tissue anchors can be implemented, such as any number between twelve and twenty. The anchors 102/202 can be repositionable and/or retrievable prior to full deployment of the implant devices as shown in FIG. 2.

Certain processes/operations can be implemented to deliver and anchor the implant devices. For example, the delivery system can be advanced toward the annulus 10. In some implementations, an outer catheter/sheath 101 can be advanced through the patient's vasculature into the right atrium 5 and through the interatrial septum until its distal end is positioned in the left atrium 2. A steerable distal end portion of the outer catheter/sheath can then be steered such that it is positioned in a desired spatial orientation within the left atrium 2. The steering procedure can be performed with the aid of imaging, such as fluoroscopy, transesophageal echo, and/or echocardiography.

Access to the right atrium 5 can be made using any suitable or desirable access path, such as through the femoral vein and/or through arterial access. It should be understood that any suitable point of origin. For example, access can be made by introduction into the femoral vein of the patient, through the inferior vena cava 19, into the right atrium 5, and into the left atrium 2 transseptally (e.g., through the fossa ovalis). In some implementations, access can be made through the basilic vein, the subclavian vein, the superior vena cava 16, and into the right atrium 5. In some implementations, access can be made via the jugular vein, the subclavian vein, the superior vena cava 16, and into the right atrium 5 and/or the left atrium 2.

Such access can be used to advance a guidewire to the target position within the left atrium 2. A relatively stiff guidewire can be utilized for the purpose of traversing the interatrial septum. The delivery system can be advanced over the guidewire into the left atrium 2. The inner catheter 103 can be placed over the anterior commissure to provide a suitable attack position for deployment of the first anchor 202a. Verification of the first anchoring location can be obtained using imaging. In some implementations, the first anchor 202a is placed close to the leaflet hinge, as anterior as possible in the annulus, close to the anterior commissure.

The first anchor 202a can be delivered and implanted using the anchor delivery drive shaft 105, which can be provided inside the catheter(s) 101, 103. The anchors 102/202 can be released after proper anchoring is checked with push-and-pull testing under imaging guidance (e.g., echocardiography, fluoroscopy). After proper anchoring of one anchor, the catheter(s) 101, 103 can be navigated (e.g., using steering knobs of a proximal handle/system, or the like) to the next anchoring point along the annulus. Such actions can be repeated until the implant catheter tip reaches the last anchoring site, where the last anchor 202b is deployed. The implant devices can then be disconnected from the delivery catheter(s) 101, 103 with the tensioning wire 210 passing from the tissue anchors 102/202 through the sheath(s) 101, 103 and the patient's anatomy to a location outside the body, wherein an adjustment tool can be advanced over the tensioning wire 210 to allow for adjustment in the tension of the wire/line 210.

The tension adjustment tool/device can be actuated/controlled to cause the tensioning wire/line 210 to be contracted. For example, rotation of a knob or other actuator associated with a handle of the tension adjustment tool/device can be executed to cause the tensioning of the wire/line 210. After and/or during tensioning, adequate reduction of heart valve regurgitation or other defect can be assessed using imaging (e.g., echo) under beating heart conditions. When the tensioning has caused the appropriate implant size/diameter to be reached, the tensioning device/tool can be used to cut and/or lock the tension in the wire/line 210, after which the non-implant instrumentation can be withdrawn from the patient, leaving the implant with the desired degree of contraction.

The anchors 202 can be anchored sequentially around all or part (e.g., 40-90%, 50-70%, etc.) of the annulus 10, which is followed by the tensioning of the wire/line 210 in order to contract the annulus 10, as shown in FIG. 2. In some implementations, tensioning the wire 210 will be sufficient to adequately reduce or eliminate regurgitation through the native valve, in which case, the method/procedure can be concluded. In some implementations, if there is still a significant amount of regurgitation at the native valve (e.g., shortly after the initial procedure steps/tensioning or after the passage of more time, such as months or years later), a replacement prosthetic valve can be deployed in the native annulus.

The implant devices can advantageously increase leaflet coaptation and/or provide support to the posterior annulus against dilation. Compared to certain surgical annuloplasty ring devices, the annuloplasty implants shown in FIG. 2, comprising a plurality of tissue anchors 202 coupled to a common tensioned wire/line 210, may advantageously provide improved flexibility to maintain the three-dimensional contour of the native annulus and some of its natural dynamics.

The tension in the wire/line 210 can be locked using a locking clamp/mechanism 282, which can be coupled to the wire/line 210 proximal to the last anchor 202b. The excess portion of the wire 210 can be cut and removed. Moreover, a pin 214 and/or similar mechanism can be configured to anchor the wire/line 210 at a starting point of the wire/line 210.

FIG. 3 illustrates an example implant system 300 in which one or more tissue anchors 302 anchor an annuloplasty tube 330 to annuloplasty tissue 10 of a mitral valve 3, in accordance with one or more implementations. The annuloplasty tube 330 can be additionally anchored using one or more pins, including a first pin 344 at or near a first tissue anchor 302a and/or a second pin 342 at or near a last tissue anchor 302b.

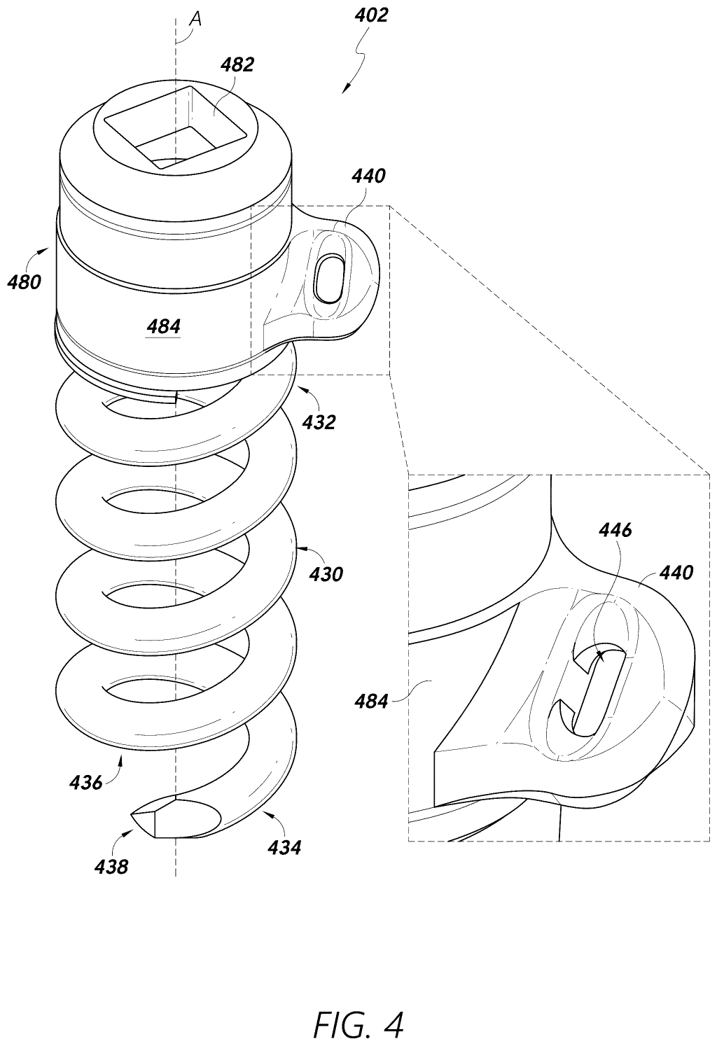

FIG. 4 illustrates an example tissue anchor 402 in accordance with one or more implementations. The tissue anchor 402 can be a component of a tissue-adjustment system and can be used for adjusting a dimension of a tissue structure. For example, the tissue-adjustment system can be an annuloplasty system and/or the tissue anchor 402 can be used in anchoring an annuloplasty structure (e.g., an annuloplasty ring, annuloplasty implant, etc.).

The anchor 402 can comprise a tissue-engaging element 430 and/or a head 480 portion. The tissue-engaging element 430 can be configured in a variety of ways. In some implementations, the tissue-engaging element 430 can have a proximal end 432 and/or a distal end 434 and/or can define a central longitudinal axis, A, of the anchor 402. At the distal end 434, the tissue-engaging element 430 can have a sharpened distal tip 438 and/or the tissue-engaging element 430 can be configured to be driven (e.g., screwed, pushed, etc.) into tissue of the subject. In some implementations, and as shown, tissue-engaging element 430 can be helical and/or can define a central lumen 436 along axis, A. Optionally, tissue-engaging element 430 can be another type of tissue-engaging element 430, such as a dart or a staple. In some implementations, the tissue-engaging element 430 can be hook-shaped, straight, angled, and/or another configuration. In some implementations, the tissue-engaging element 430 can include barbs or barbed portions to hold the tissue-engaging element in tissue.

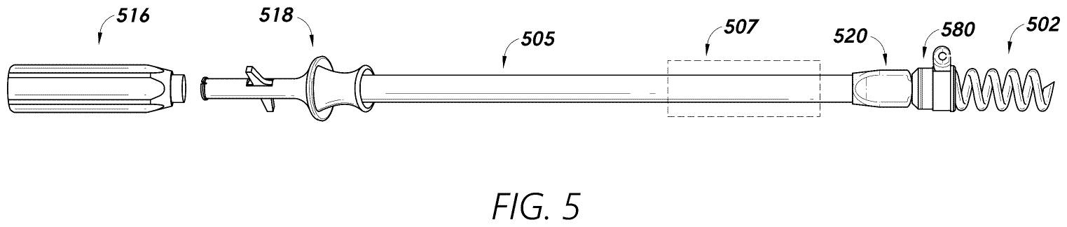

Tissue-engaging element 430 can have any suitable lateral width. For implementations in which tissue-engaging element 430 is helical (e.g., as shown in FIG. 4), width can be an outer diameter of the helix. The head 480 of the anchor 402 can be coupled to proximal end 432 of tissue-engaging element 430 and/or can comprise a driver interface 482 and/or an eyelet 440 that can define an aperture 446 therethrough. Driver interface 482 can be configured to be reversibly engaged by an anchor driver (see, e.g., FIG. 5). In some implementations, an anchor driver can comprise an elongate and/or flexible shaft and/or a driver head coupled to a distal end of the shaft. Driver head can be a component of the anchor driver that reversibly engages driver interface 482. Driver interface 482 can be rigidly coupled to tissue-engaging element 430.

In some implementations, and as shown, driver interface 482 can be disposed on central longitudinal axis, A, and eyelet 440 can be disposed laterally from axis, A.

In some implementations, the anchor 402 (e.g., connector or eyelet 440 thereof) can be configured to facilitate sliding of the anchor 402 along a wire (or sliding of the wire through the anchor 402) while the anchor 402 is aligned with the wire (e.g., while axis, A, is parallel with the wire). The anchor 402 (e.g., connector or eyelet 440 thereof) is configured to facilitate sliding of the anchor 402 along the wire (or sliding of the wire through the anchor 402) while the anchor 402 is oriented orthogonal to the wire (i.e., while axis, A, is orthogonal to the wire). This can be achieved at least partly due to the shape and dimensions of connector or eyelet 440.

Eyelet 440 can define aperture 446 on an aperture plane and/or can be mounted such that the aperture plane is slanted at a fixed angle (e.g., approximal 30-60 degrees, such as at 45 degrees, from axis, A).

In some implementations, eyelet 440 is shaped to define (i) a first clear straight pathway through aperture 446 along a first line that is parallel to axis, A, and (ii) a second clear straight pathway through the aperture 446 along a second line that is orthogonal to the first line. This shape can advantageously allow sliding of eyelet 440 along a wire in either of these mutually orthogonal orientations. The shape of eyelet 440 can similarly facilitate its sliding along a wire when in an orientation that is partway between these mutually orthogonal orientations.

Despite the actual shape of aperture 446, described hereinabove, eyelet 440 can be shaped and dimensioned such that both (i) when viewed along the first line (i.e., a first view-line) that is parallel to axis, A, and (ii) when viewed along the second line (i.e., a second view-line) that is orthogonal to the first view line, aperture 446 appears to be circular. This shape can advantageously allow smooth sliding of eyelet 440 along a wire in either of these orientations (and typically also in a continuum of orientations therebetween). Thus, these view lines can be considered to be first and second slide axes of anchor 402 (e.g., of eyelet 440 thereof). This shape can advantageously allow such sliding even when the wire is more than 50% as great (e.g., more than 70% as great, such as more than 90% as great) as the diameter of the apparent circular shape of aperture 446.

To further facilitate smooth sliding of a wire through aperture 446, eyelet 440 defines a beveled rim around the aperture. In some implementations, and as shown, beveling can be greater on long axis than on short axis (i.e., at the sides of the aperture 446). In some implementations, on each face of eyelet 440 (i.e., on each side of aperture 446), the eyelet 440 defines a bathtub-shaped cavity, with the bottoms of the bathtubs meeting to form aperture 446.

In some implementations, and as shown, eyelet 440 is mounted to be revolvable or rotatable around axis, A, while aperture plane remains slanted at its fixed angle with respect to the central longitudinal axis. Optionally, eyelet 440 can be revolvable and/or rotatable around another axis. For example, head 480 can comprise a ring 484 on which eyelet 440 is mounted. Ring 484 can circumscribe and/or can be rotatable about axis, A (e.g., by being rotatably coupled to tissue-engaging element 430, such as by being rotatably coupled to another component of head 480, such as the driver interface 482, that is fixedly coupled to the tissue-engaging element).

In some implementations in which tissue-engaging element 430 is helical, on the side of anchor 402 on which eyelet 440 is disposed, the helix of the tissue-engaging element 430 slants in the same direction as aperture plane with respect to axis, A. However, the lead angle of the helix of tissue-engaging element 430 can be different from angle of aperture plane.

As described hereinabove, anchor 402 (e.g., eyelet 440 thereof) can be configured to facilitate sliding of the anchor 402 along a wire (or sliding of the wire through the anchor 402) while the anchor 402 is aligned with the wire (e.g., while axis, A, is parallel with the wire). This can facilitate transcatheter advancement of anchor 402 along the wire. As also described hereinabove, anchor 402 (e.g., eyelet 440 thereof) is configured to facilitate sliding of the anchor 402 along the wire (or sliding of the wire through the anchor) while the anchor 402 is oriented orthogonal to the wire (i.e., while axis, A, is orthogonal to the wire). This can be useful for implementations in which the wire is tensioned after implantation in order to adjust anatomical dimensions, such as annuloplasty.

FIG. 5 illustrates an example system (e.g., delivery system, anchor delivery system, implant delivery system, etc.) for delivering one or more tissue anchors 502 to a target tissue site in accordance with one or more implementations. The delivery system can comprise an elongate shaft 505 which can comprise one or more flex portions 507 comprising any of the various features described herein with respect to flex portions. The elongate shaft 505 can extend between a drive handle 518 at a proximal side and/or end of the shaft 505 and/or a driver 520 at a distal side and/or end of the shaft 505. The driver 520 can be configured to interface with a head 580 portion and/or drive interface of the tissue anchor 502. For example, the driver 520 can comprise a generally rectangular protrusion configured to mate with a similarly rectangular interface of the tissue anchor 502.

The drive handle 518 can be coupled to a torque limiter 516 configured to prevent and/or inhibit over-torquing of the elongate shaft 505. For example, the torque limiter 516 and/or drive handle 518 can be configured to be twisted to cause twisting and/or torquing of the elongate shaft 505 and/or tissue anchor 502. The torque limiter 516 can be configured to prevent and/or inhibit twisting of the shaft at or above a threshold torque amount.

In some implementations, the torque limiter 516 can comprise various components, which can include dimples, ball bearings, and/or washers pressed against a ball bearing. The torque limiter 516 can be calibrated with a screw and/or similar mechanism. When the torque limiter is rotated, a ball bearing can be configured to become dislodged when torque exceeds a predetermined amount and/or can rotate freely to not engage the anchor drive.

The elongate shaft 505 can be an inner shaft of a delivery system and/or can be configured for delivery at least partially through an outer shaft. For example, at least a portion of the elongate shaft 505 can be configured to be extended through a catheter and/or other outer shaft during delivery into a patient. The outer shaft can be configured to at least partially enclose the elongate shaft 505.

As shown in FIG. 5, a flex portion 507 can be situated at or near a distal end of the elongate shaft 505. For example, the flex portion 507 can be nearer to the driver 520 than to the handle 518. However, the flex portion 507 can be situated at any position along the elongate shaft 505.

In some implementations, the flex portion 507 can be an extension of the elongate shaft 505. For example, the flex portion 507 can represent a portion of the elongate shaft 505 comprising one or more features configured to facilitate a state change of the elongate shaft 505 and/or flex portion 507. In some implementations, the flex portion 507 can comprise a cut through at least a portion of the elongate shaft 505 and/or can comprise one or more components (e.g., springs, coils, and/or wires) configured to interconnect separate and/or disconnected portions of the elongate shaft 505. The flex portion 507 can comprise one or more features configured to cause a state change of the elongate shaft 505 in response to the tissue anchor 502 being fully embedded in tissue and/or in response to twisting and/or torquing force of the elongate shaft 505 meeting and/or exceeding a threshold amount of force. In some implementations, the flex portion 507 can be configured to have a first state during delivery and/or to assume a second state during a driving and/or delivery process of the tissue anchor 502 (e.g., once the tissue anchor 502 has been fully driven into the tissue and additional twisting force is applied). For example, once the tissue anchor 502 is fully embedded in tissue while the driver 520 is mated to the head 580, additional twisting force at the elongate shaft 505 and/or handle 518 can cause the flex portion 507 and/or elongate shaft 505 to move from a first state (e.g., a generally linear state) to a second state (e.g., a bent state).

The flex portion 507 can comprise one or more features configured to facilitate delivery and/or driving of the elongate shaft 505 and/or tissue anchor 502. For example, the flex portion 507 can be configured to have a generally flexible state during delivery and/or to assume a generally rigid state prior to driving of the tissue anchor 502 into a target tissue site. In some implementations, one or more pull wires can extend at least partially through the elongate shaft 505 and/or flex portion 507 (e.g., through apertures in walls of the elongate shaft 505 and/or flex portion 507) to facilitate movement of the flex portion 507 and/or elongate shaft from a first state to a second state and/or vice versa.

While the flex portion 507 is shown in FIG. 5 as a portion of an elongate shaft 505 configured to drive one or more tissue anchors 502, flex portions 507 can be components of other shafts. For example, an outer shaft configured to receive and/or facilitate delivery of an elongate shaft 505 can comprise one or more flex portions 507 in various locations.

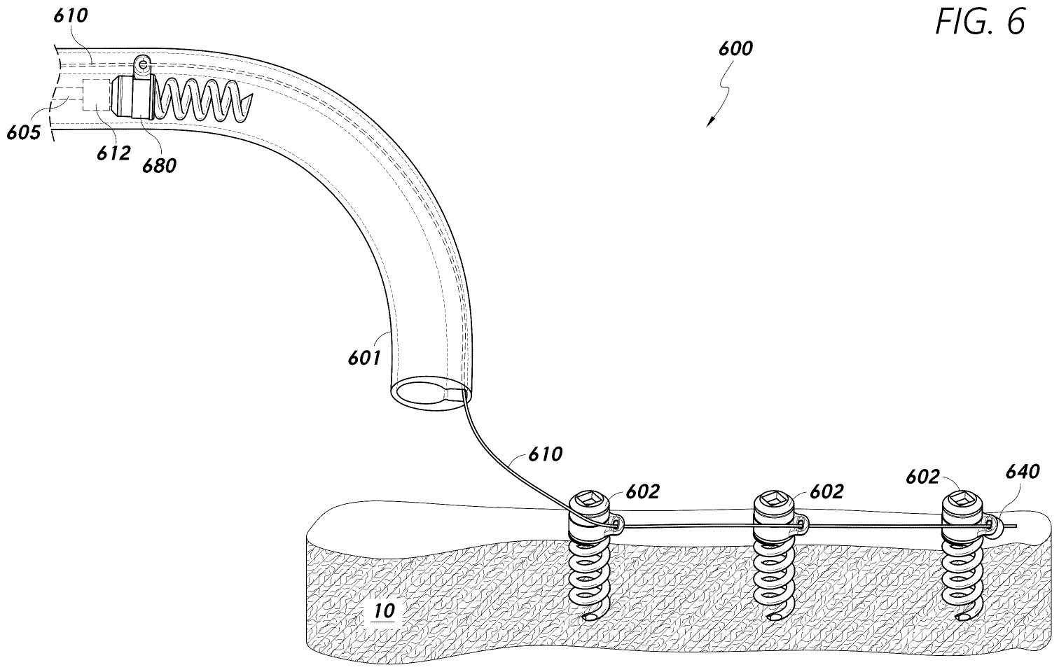

FIG. 6 illustrates a system 600 (e.g., an anchor delivery system, implant delivery system, etc.) for delivering one or more tissue anchors 602 into tissue 10, in accordance with one or more implementations. The system 600 can comprise a catheter 601 (e.g., an elongate shaft and/or outer shaft) and/or a shaft 605 (e.g., an elongate shaft and/or inner shaft) configured to be extended through the catheter 601. The shaft 605 can comprise an anchor driver 612. The system 600 can be configured delivery of one or more tissue anchors 602 and/or a tether 610 (e.g., a wire and/or cord) on which the tissue anchors 602 are threaded (e.g., through one or more eyelets 640 of the tissue anchors 602). As described in more detail hereinbelow, during implantation only a distal portion of tether 610 may remain implanted in the subject, while a proximal portion of the tether remains attached to the delivery system 600.

Tissue anchors 602 can be distributed in a series along tether 610, and delivery system 600 can be used to implant the one or more tissue anchors 602 by anchor driver 612 being used, for each of anchors 602 consecutively, to advance the anchor 602 distally into the subject and to anchor the anchor 602 to internal tissue of the subject. For example, and as shown, the one or more tissue anchors 602 and/or tether 610 can comprise an annuloplasty implant, implanted by distributing anchors 602 around at least a portion of an annulus of a native heart valve of the subject, such as the mitral or tricuspid valve. Further in some implementations, a distal end of tether 610 can be advanced distally into the subject along with the first anchor, and subsequent anchors 602 can be advanced by sliding them distally along the tether 610. In some implementations, system 600 and/or techniques described for use therewith are used in combination with one or more of the systems and/or techniques described in U.S. Provisional Patent Application 62/949,392 to Kasher et al., filed on Dec. 17, 2019, and entitled ANNULOPLASTY AND TISSUE ANCHOR TECHNOLOGIES,” which is incorporated herein by reference.

The catheter 601 can be configured to be advanced into the subject. In some implementations in which the implant is an annuloplasty implant, and as shown, catheter 601 can be a transluminally (e.g., transfemorally) advanceable catheter. The catheter 601 can extend from an extracorporeal unit (e.g., an extracorporeal unit), configured to remain outside the body of the subject. In some implementations, extracorporeal unit defines, or is coupled to, a handle of the device. A tissue anchor 602 can comprise various features (e.g., a head 680 portion) to allow the tissue anchor 602 to interface with and/or attach to the driver 612 of the shaft 605.

Extracorporeal unit can comprise a tensioner that comprises a winch that facilitates reducing slack on tether 610 during sliding of anchors 602 distally along the tether 610. Reducing slack can advantageously reduce a likelihood of tether 610 becoming twisted or entangled, or of inadvertent engagement of the tether 610 with the anchor 602 being delivered. Reducing slack using a winch, rather than by a human operator manually pulling on a proximal end of the tether 610, can further advantageously provide greater control over the magnitude and consistency of tension applied to the tether 610, and may further advantageously reduce the number of human operators required.

FIG. 7 illustrates anchoring of one or more tissue anchors to tissue 10 in accordance with one or more implementations. In FIG. 7, at least the right-most anchor has already been successfully anchored, and the left-most anchor has not yet been successfully anchored. Therefore, the behavior of the anchors (including movement of the protrusion with respect to head 780) can be understood from these figures by comparing the left anchor to the other anchors.

In some implementations, a catheter 701 and/or similar device can be configured to deliver the shaft 705 and/or one or more anchors 702 via a lumen of the catheter 701. A tether 710 can be configured to interconnect the various tissue anchors 702 and/or can pass through one or more eyelets, which can extend from head 780 portions of the tissue anchors 702. A driver 712 of the shaft 705 can be configured to interface with the head 780 portions to carry and/or drive the tissue anchors 702. The shaft 705 can be configured to drive the tissue-engaging elements 730 of the anchors 702 into the tissue until a proximal end 734 of the tissue-engaging element 730 and/or a washer 732 or similar mechanism of the anchor 702 between the tissue-engaging element 730 and the head 780 reaches the tissue 10. The catheter 701 and/or the shaft 705 can be portions/components of an anchor delivery system (e.g., of any of the anchor delivery systems described herein).

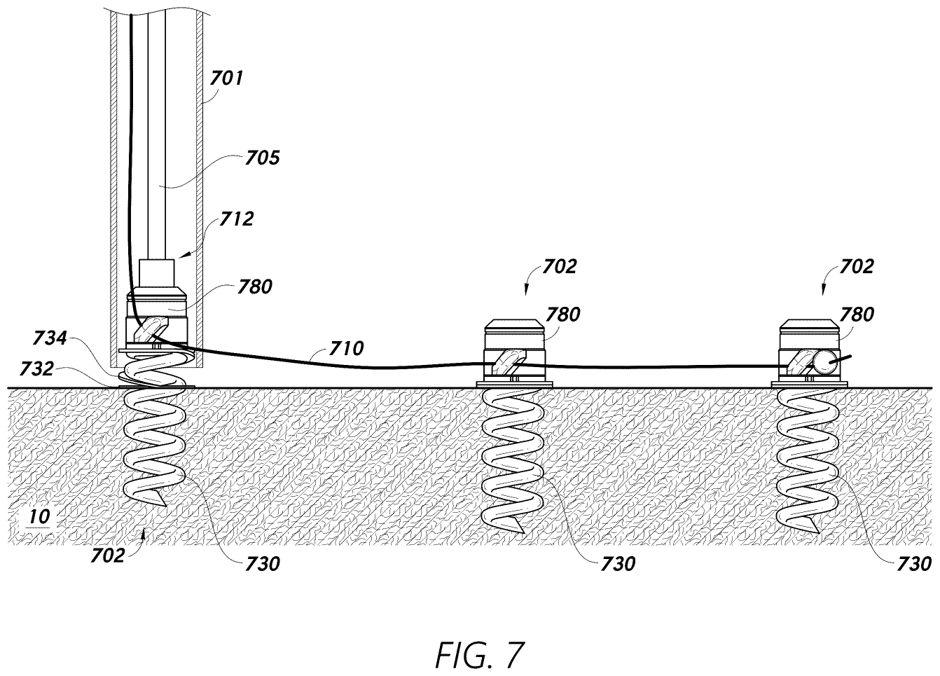

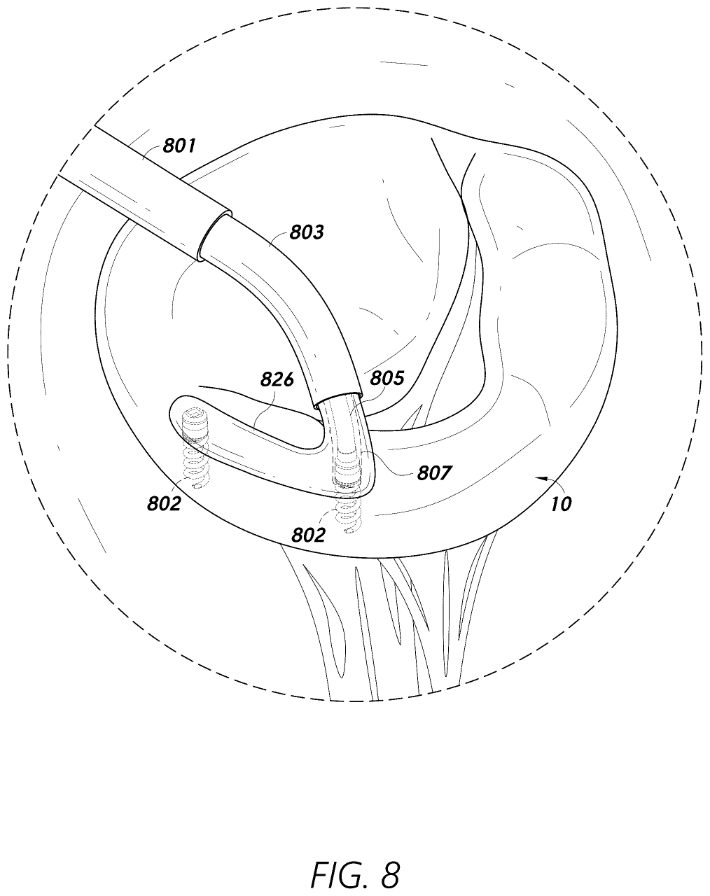

FIG. 8 illustrates delivery of one or more tissue anchors 802 into tissue 10 in accordance with one or more implementations. A delivery system (e.g., an anchor delivery system, etc.) can include, among other things, an outer catheter 801, an inner catheter 803, and/or a channel 807 through which a drive shaft 805 can pass. In some implementations, the one or more tissue anchors 802 can be configured to anchor at least partially through an elongate sleeve 826. A portion of a lateral wall of sleeve 826 aligns against the tissue 10 in a manner in which a surface of the portion of the lateral wall is disposed in parallel with the planar surface of the tissue 10. Additionally, a distal end of a channel 807 extending around a shaft 805 can be configured to flatten the portion of the lateral wall against the tissue 10 of annulus in a manner in which channel 807 sandwiches the portion of the lateral wall between the distal end of implant-decoupling channel and the portion of the tissue 10 of annulus at the planar surface into which the tissue anchors 802 are implanted. In such a manner, the portion of the lateral wall being anchored lies flat against the tissue 10 of annulus (parallel with the planar surface thereof), while the remaining portion of the tubular lateral wall is disposed substantially perpendicularly with respect to the portion of the tissue into which the tissue anchor 802 is implanted.

In some implementations, different tissue anchors 802 can be deployed through different portions and/or sides of the sleeve 826. For example, a first tissue anchor 802 can be deployed through an end wall of the sleeve 826 and/or a second tissue anchor can be deployed through a lateral wall of the sleeve 826. However, multiple tissue anchors 802 can extend in a substantially same direction and/or into a common, substantially planar surface of a valve annulus. In some implementations, tissue anchors 802 can be disposed with respect to each other at an angle of between 0 and 45 degrees, e.g., between 0 and 30 degrees, e.g., between 0 and 20 degrees.

In some implementations, a maximum distance between a first tissue anchor 802 and a point of anchoring of a second tissue anchor 802 can be provided by a length of the sleeve 826 that has been decoupled from the portion of channel 807 (e.g., by the distance that channel 807 has been retracted from sleeve 826, e.g., between 3 and 15 mm, e.g., 8 mm). That is, in some implementations, a second tissue anchor 802 can be placed anywhere within a circle having a radius that equals the distance between a first anchor 802 and a second anchor 802, centered on the first tissue anchor 802. In some implementations, sleeve 826 can serve as a constraining member (e.g., a tether) that can be used to facilitate positioning of a second tissue anchor 802. The distance between the tissue anchors 802 can be set by the operating physician retracting channel 807 from sleeve 826 by a particular distance.

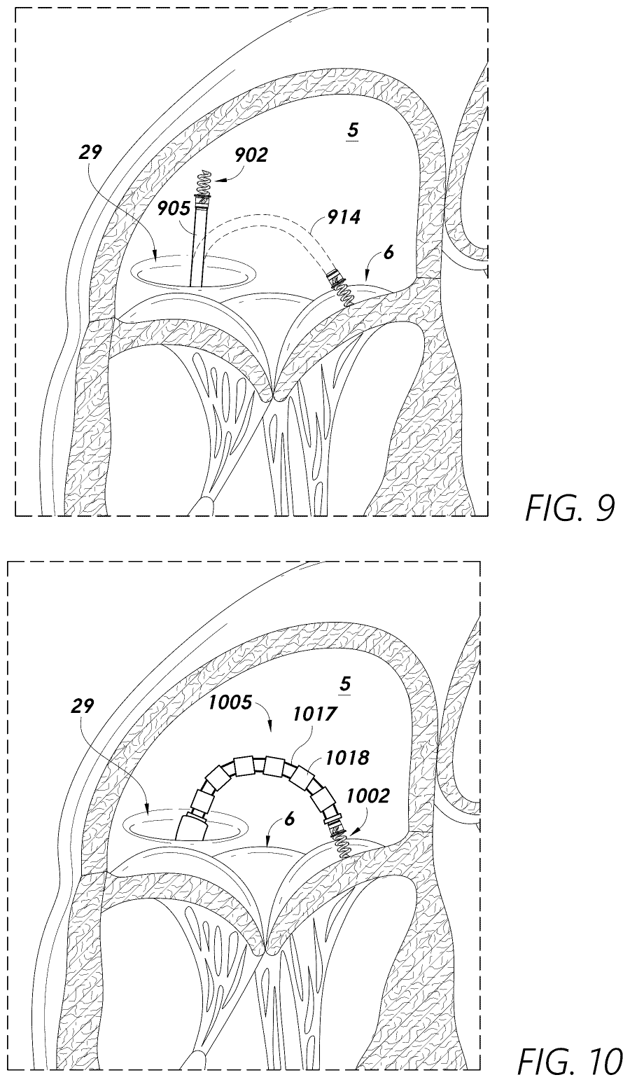

FIG. 9 illustrates delivery of a delivery shaft 905 into a chamber (e.g., a right atrium 5) of a heart in accordance with one or more implementations. For example, the delivery shaft 905 can be delivered upwards via the inferior vena cava 29 into the right atrium 5. In some implementations, one or more delivery shafts 905 can be configured for delivery via other pathways, including via the superior vena cava into the right atrium 5. Delivery via the inferior vena cava 29 is shown for illustrative purposes and the devices and/or methods described herein can be applicable to other delivery procedures and/or other anatomies. The delivery shaft 905 can be a portion/component of an anchor delivery system (e.g., of any of the anchor delivery systems described herein).

The position of the tricuspid valve 6 relative to the inferior vena cava 29 and/or superior vena cava may require curvature of delivery shafts 905 delivered via the inferior vena cava 29 and/or superior vena cava. For example, an opening of the inferior vena cava 29 into the right atrium 5 may be at a relatively common plane and/or adjacent to the tricuspid valve 6 along a lower portion of the right atrium 5. As the delivery shaft 905 exits the inferior vena cava 29 into the right atrium 5, the delivery shaft 905 can be pointed toward an upper area of the right atrium 5. In order to reach the tricuspid valve 6, the delivery shaft 905 may be required to bend approximately to a U-shape to redirect a distal end of the delivery shaft 905 to the tricuspid valve 6, as shown by a target orientation 914 in FIG. 9. In cases in which the delivery shaft 905 is delivered via the superior vena cava, the delivery shaft 905 may similarly be required to bend to reach the tricuspid valve 6. Accordingly, it may be advantageous for the delivery shaft 905 to be at least partially flexible to enable access of the tricuspid valve 6 via the inferior vena cava 29 and/or superior vena cava, and/or to enable access of other anatomies via other pathways.

The delivery shaft 905 can be configured for delivery of one or more anchors 902, which can be configured to extend through a lumen of the delivery shaft 905 and/or from a distal end of the delivery shaft 905. The delivery shaft 905 can be configured to bend to cause one or more anchors 902 at a distal end of the delivery shaft 905 to be positioned at or around the tricuspid valve 6 and/or another valve.

FIG. 10 illustrates an example flexible delivery shaft 1005 configured for delivery of one or more anchors 1002 at various portions of anatomy, which can include at or around a tricuspid valve 6, mitral valve, and/or another valve. While FIG. 10 illustrates one implementation delivery shaft 1005 comprising a flex portion including tabs 1017 interconnecting segments 1018 of the delivery shaft 1005 and/or flex portion for illustrative purposes, other implementations described herein can be used to allow for improved flexibility while delivering one or more anchors 1002 to target anatomies. The delivery shaft 1005 can be a portion/component of an anchor delivery system (e.g., of any of the anchor delivery systems described herein).

In some implementations, the delivery shaft 1005 can comprise multiple portions and/or segments 1018, which can comprise one or more flex portions and/or one or more non-flex portions. A flex portion can include any portion configured to provide improved flexibility of the delivery shaft 1005 and/or configured to facilitate state changes of the shaft 1005. For example, a flex portion can include a portion of the delivery shaft comprising one or more tabs 1017 and/or segments 1018, as shown in FIG. 10. In some implementations, the delivery shaft 1005 can comprise at least one flex portion at or near a distal end and/or distal portion of the delivery shaft 1005. For example, a flex portion can be configured to enable bending of a distal portion of the delivery shaft 1005 to allow one or more anchors 1002 to be deployed from the tip portion at a target location.

A flex portion can be configured to selectively provide improved flexibility and/or rigidity. In some implementations, a flex portion can be transitioned, transformed, and/or modified from a first state providing improved flexibility to a second state providing improved rigidity, and/or from a second state providing improved rigidity to a first state providing improved flexibility. For example, the flex portion can be configured to provide improved flexibility during delivery to a target location and/or can be modified and/or moved to provide improved rigidity during insertion of one or more anchors 1002 into a target tissue location (e.g., tissue around a valve). Moreover, the flex portion can be configured to be transitioned/modified to provide improved flexibility to facilitate removal of the delivery shaft from the body.

In some implementations, one or more flex portions can be modifiable by surgeons and/or otherwise during a procedure. For example, a surgeon can use one or more pull wires and/or similar devices (e.g., actuate a knob, button, or other control to tension pull wires, etc.) to cause a transformation of a flex portion from one state (e.g., a flexible and/or rigid state) to another state (e.g., a rigid and/or flexible state). In the flexible and/or rigid state, the shaft 1005 and/or flex portion can be configured to bend to the “U” shape shown in FIG. 10.

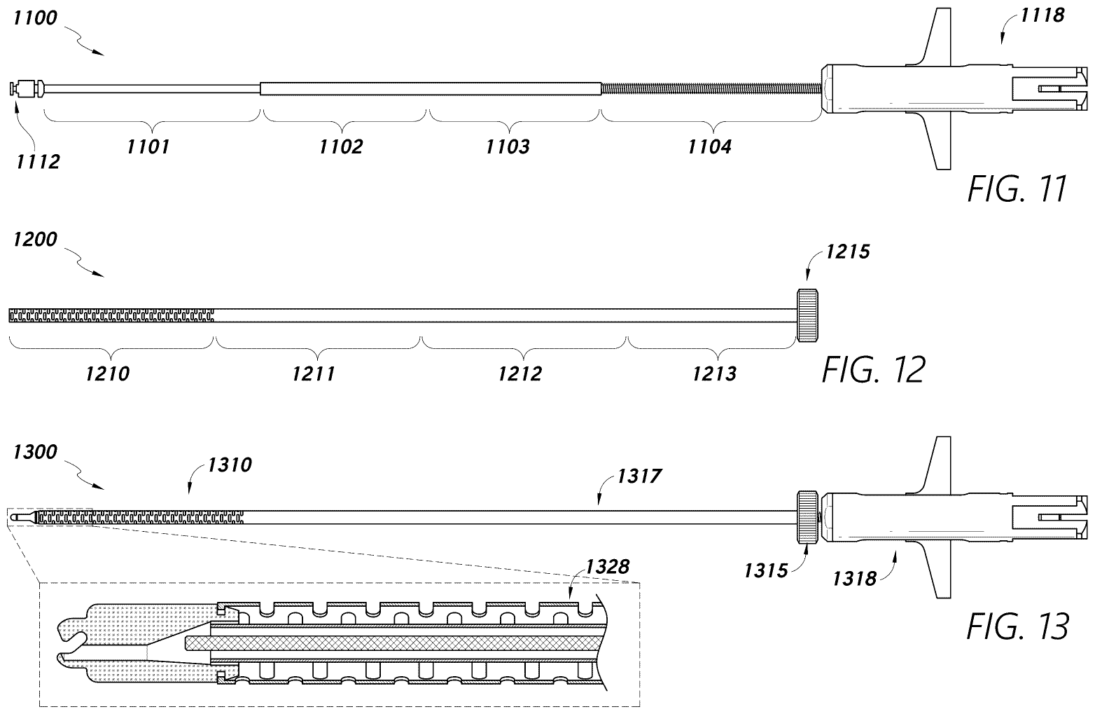

FIG. 11 illustrates an example assembly 1100 for driving one or more anchors into target tissue, in accordance with one or more implementations. The assembly 1100 can comprise a catheter and/or outer shaft (see FIGS. 12 and 13) and/or can be configured for use alone or in combination with other assemblies. The assembly 1100 can be configured as an anchor delivery system or can be a portion of an anchor delivery system (e.g., of any of the anchor delivery systems described herein).

In some implementations, the assembly 1100 can comprise multiple distinct and/or interconnected segments. At a distal end of the assembly 1100, the assembly 1100 can comprise a gripper 1112 configured to grip onto and/or otherwise engage one or more tissue anchors. For example, the gripper 1112 can be configured to fit into a notch and/or aperture of a tissue anchor head and/or can be configured to fit at least partially around the tissue anchor. In some implementations, the assembly 1100 can comprise a groove cut around the gripper 1112 and/or configured to mate with protrusions of another catheter and/or assembly.

The assembly 1100 can be at least partially flexible to facilitate delivery of the assembly 1100 through one or more pathways through a body and/or to a target tissue site. In some implementations, the assembly 1100 can have varying flexibility along a length of the assembly 1100. For example, the assembly 1100 can comprise a generally flexible segment 1101 at or near a distal end of the assembly 1100, a generally rigid segment 1103 at or near a proximal end of the assembly 1100, and/or a semi-flexible segment 1102 between the generally flexible segment 1101 and the generally rigid segment 1103. In some implementations, the generally flexible segment 1101, semi-flexible segment 1102, and/or generally rigid segment 1103 can be aligned longitudinally and/or can form a shaft extending between a handle 1118 of a device and the gripper 1112. The flexible segment 1101 can have a smaller width and/or diameter than the semi-flexible segment 1102 and/or the generally rigid segment 1103, as shown in FIG. 11. However, the flexible segment 1101 can optionally have a generally equivalent width and/or diameter to the semi-flexible segment 1102 and/or the generally rigid segment 1103. The flexible segment 1101 can be at least somewhat more flexible than the semi-flexible segment 1102 and/or the generally rigid segment. In some implementations, increased flexibility of the flexible segment 1101 and/or semi-flexible segment 1102 relative to the generally rigid segment 1103 can be due at least in part to being at least partially composed of different materials and/or having smaller widths, diameters, and/or wall thicknesses.

In some implementations, the assembly 1100 can further comprise a threaded segment 1104 situated at least partially between the generally rigid segment 1103 and the handle 1118. The flexible segment 1101, semi-flexible segment 1102, rigid segment 1103, and/or threaded segment 1104 can be configured to fit at least partially within an outer catheter (e.g., the assembly 1200 of FIG. 12). Accordingly, the various segments of the assembly 1100 can be generally thin to allow the segments to fit within an outer catheter.

In some implementations, the assembly 1100 and/or shaft of the assembly 1100 can be generally incompressible. However, the assembly 1100 can have some flexibility. For example, the flexible segment 1101 can have an approximately 10 mm bend radius, the semi-flexible segment 1102 can have an approximately 25 mm bend radius, and/or the rigid segment 1103 can have a relatively high bend radius. The flexible segment 1101 can have a length of approximately 120 mm, the semi-flexible segment 1102 can have a length of approximately 988 mm, and/or the rigid segment 1103 can have a length of approximately 620 mm.

The shaft of the assembly 1100 can have an inner diameter of approximately 0.5 mm or greater. The outer diameter can be sufficiently small to fit inside a lumen of a shaft of an outer assembly (e.g., the assembly 1200 illustrated in FIG. 12). The shaft can have a default straight and/or linear form.

In some implementations, the assembly 1100 can comprise one or more flex portions. For example, the flexible segment 1101 can comprise a cut separating the flexible segment 1101 into disconnected portions and/or can comprise one or more coils and/or wires configured to interconnect the disconnected portions. The flex portion can be configured to bend and/or the disconnected portions of the flex portion can be configured to become offset in response to twisting of the handle 1118 and/or various shafts of the assembly 1100.

FIG. 12 illustrates at least a portion of an assembly 1200 for delivering and/or driving one or more tissue anchors to target tissue locations, in accordance with one or more implementations. In some implementations, the assembly 1200 can be configured for use with one or more other devices. For example, the assembly 1200 can be configured for use as an outer catheter/sheath and/or can be configured to receive an inner catheter/shaft and/or assembly (e.g., the various segments of the assembly 1100 of FIG. 11). The assembly 1200 can be configured as an anchor delivery system or can be a portion of an anchor delivery system (e.g., of any of the anchor delivery systems described herein).

In some implementations, the assembly 1200 can comprise multiple distinct and/or interconnected segments and/or portions, which can include a flex portion 1210, a generally flexible segment 1211, a semi-flexible segment 1212, and/or a generally rigid segment 1213. The flex portion 1210 can be at least partially compressible from an expanded form (e.g., during delivery) to a compressed form (e.g., following delivery and/or while driving one or more tissue anchors into tissue).

The assembly 1200 can comprise one or more controls or control features, which can include a knob 1215, button, switch, slider, and/or similar mechanism. For example, a knob 1215 can be situated at a proximal end of the assembly 1200. In some implementations, the knob 1215 can be configured to control compression, expansion, and/or other transformation of the flex portion 1210. For example, as the knob 1215 is rotated in a first direction (e.g., counterclockwise), the flex portion 1210 can be compressed to become more rigid. Similarly, as the knob 1215 is rotated in a second direction (e.g., clockwise), the flex portion 1210 can be decompressed and/or can become more flexible. By compressing, the flex portion 1210 can be configured to resist a compressive load applied when a wire passing through the assembly 1200 is put under tension.

In some implementations, the assembly 1200 can be configured to mate with various features and/or devices. For example, the assembly 1200 can comprise one or more protrusions (e.g., at the distal end of the assembly 1200) configured to mate with a corresponding groove of the assembly 1100 of FIG. 11. In this way, the assembly 1200 and/or other assemblies and/or catheters can be held together to prevent and/or inhibit shifting relative to each other.

The various segments of the assembly 1200 can form a tubular shaft configured to receive and/or convey one or more wires and/or tissue anchors. In some implementations, the various segments can have varying flexibility to facilitate delivery of the assembly 1200 through various anatomical pathways. The various segments of the assembly 1200 can be at least partially composed of different materials and/or can comprise at least partially different wall thicknesses and/or lumen widths and/or diameters with respect to each other.

The flex portion 1210 can be at least partially compressible and/or the other portions of the shaft of the assembly 1200 can be generally incompressible. In some implementations, the flex portion 1210 can have a length of approximately 30 mm and/or the generally flexible segment 1211 can have a length of approximately 90 mm. The semi-flexible segment 1212 can have a length of approximately 988 mm and/or the rigid segment 1213 can have a length of approximately 620 mm. In some implementations, the flex portion 1210 and/or the generally flexible segment 1211 can be configured to fit within the generally flexible segment 1101 of the assembly 1100 of FIG. 11. The semi-flexible segment 1212 can be configured to fit at least partially within the semi-flexible segment 1102 of the assembly 1100 of FIG. 11. The rigid segment 1213 can be configured to fit at least partially within the rigid segment 1103 and/or braided segment 1104 of the assembly 1100 of FIG. 11.

In some implementations, the shaft of the assembly 1200 can have an inner diameter that is sufficiently large to fit the outer diameter of the shaft of the assembly 1100 of FIG. 11 into the lumen of the assembly 1200. The shaft of the assembly 1200 can have an outer diameter of approximately 2.41 mm or less.

FIG. 13 illustrates an example assembly 1300 configured for delivery and/or driving of various tissue anchors to target tissue locations, in accordance with one or more implementations. In some implementations, the assembly 1300 can comprise an inner catheter and/or assembly (e.g., the assembly 1100 of FIG. 11) and/or an outer catheter and/or assembly (e.g., the assembly 1200 of FIG. 12). The outer assembly can comprise a shaft 1317 configured to receive and/or extend at least partially over a corresponding shaft of the inner assembly. The assembly 1300 can comprise a flex portion 1310 configured to facilitate state changes of the outer assembly and/or inner assembly. For example, the flex portion 1310 can be situated at a distal portion of the shaft 1317 and/or can be configured to be transitioned or modified from a flexible state to a rigid state. In some implementations, the assembly 1300 can comprise a knob 1315 and/or similar mechanism configured to selectively control a transition of the flex portion 1310 from a first state (e.g., the flexible state) to a second state (e.g., the rigid state) and/or vice versa. The assembly 1300 can be configured as an anchor delivery system or can be a portion of an anchor delivery system (e.g., of any of the anchor delivery systems described herein).

The assembly 1300 can comprise a handle 1318 and/or similar device configured for handling by surgeons for controlled delivery of the shaft 1317 and/or other portions of the assembly 1300 into and/or through a human body. In some implementations, the knob 1315 and/or other control mechanism can be situated at or near the handle 1318.

The flex portion 1310 can comprise any suitable state-change features as described herein. For example, the assembly 1300 can comprise one or more pull wires extending at least partially through the flex portion 1310 to control and/or facilitate state changes of the flex portion 1310. The one or more pull wires can be controlled at least in part using the handle 1318 and/or knob 1315. While the flex portion 1310 is illustrated as comprising a series of interlocking plates 1328 and/or teeth, the flex portion 1310 can comprise alternative and/or additional features. For example, the flex portion 1310 can comprise one or more springs and/or networks of wires.

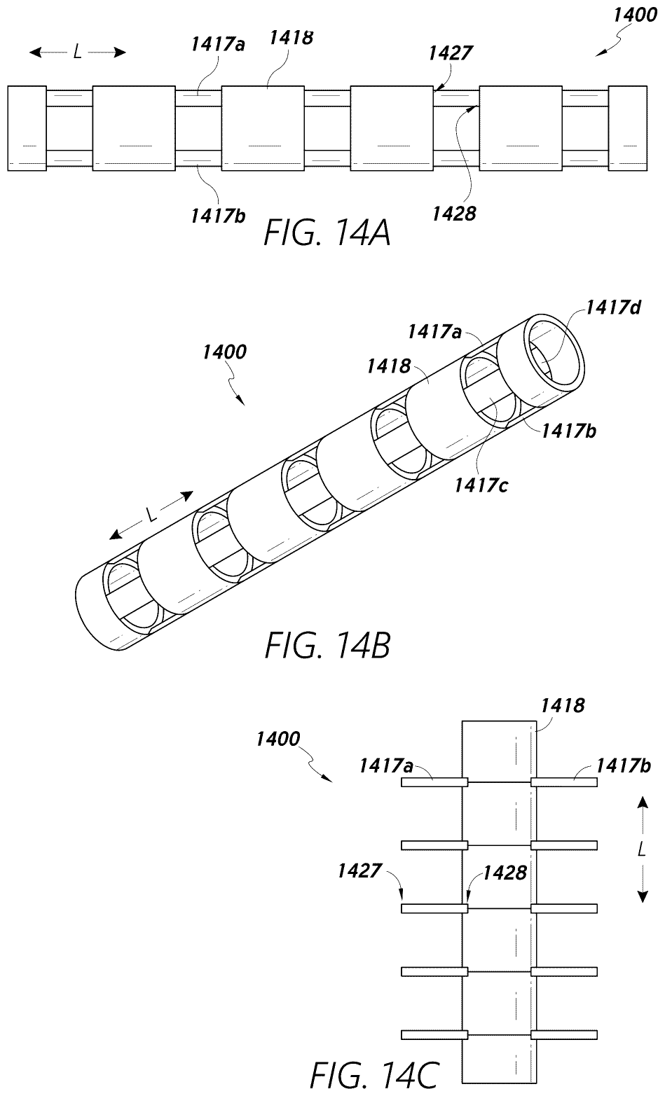

FIGS. 14A-14C illustrate an example flex portion 1400 of one or more shafts of a tissue anchor delivery assembly in accordance with one or more implementations described herein. In some implementations, a delivery assembly can comprise a shaft having a generally tubular form and/or comprising an inner lumen. The inner lumen can be configured to receive and/or facilitate conveyance of one or more wires, delivery systems, and/or medical implants (e.g., tissue anchors). The flex portion 1400 can similarly have a generally tubular form and/or can comprise an inner lumen. The flex portion 1400 can be a portion/component of an anchor delivery system (e.g., of any of the anchor delivery systems described herein).

The flex portion 1400 can comprise a network of one or more rings 1418 interconnected by one or more tabs 1417. The one or more tabs 1417 can be configured to extend between multiple rings 1418 of the flex portion 1400. In some implementations, the rings 1418 can have a circular and/or cylindrical form. The one or more tabs 1417 can have any suitable form, which can include a rectangular shape. The flex portion 1400 can comprise any number of tabs 1417 and/or any number of tabs 1417 can be used to bridge two rings 1418 of the flex portion 1400. For example, the flex portion 1400 can comprise two tabs 1417 or four tabs 1417 between each pair of rings 1418.

In some implementations, the flex portion 1400 can comprise pairs of tabs 1417 situated approximately across from each other across a lumen of the flex portion 1400. For example, as shown in FIG. 14A, a set of tabs 1417 is shown in which a first tab 1417a can be situated approximately across from a second tab 1417b. The flex portion 1400 can comprise multiple pairs of tabs 1417 bridging two rings 1418. For example, as shown in FIG. 14B, a set of tabs 1417 is shown in which the flex portion can comprise a first tab 1417a approximately across from a second tab 1417b and/or can comprise a third tab 1417c approximately across from a fourth tab 1417d.

The flex portion 1400 can have any suitable length, L. In some implementations, the flex portion 1400 can comprise any number of rings 1418 and/or tabs 1417 extending along the length of the flex portion 1400. While the flex portion 1400 is shown comprising six rings 1418 and/or five sets of tabs 1417, the flex portion can comprise any number of rings 1418 and/or sets of tabs 1417.

In some implementations, the flex portion 1400 can be controllably moved between an expanded and/or flexible state (shown, e.g., in FIGS. 14A and 14B) and a collapsed and/or rigid state (shown, e.g., in FIG. 4C). In the expanded and/or flexible state, the one or more tabs 1417 can be configured to be attached to rings 1418 at either side of the tabs 1417 (e.g., at a first side 1427 and at a second side 1428 of the tab 1417). The one or more tabs 1417 can be configured to at least partially detach from at least one of the rings 1418. For example, as shown in FIG. 14C, a first side 1427 of a tab 1417 can be disconnected from any of the rings 1418 while a second side 1428 of the tab 1417 remains connected to one or more rings 1418. Accordingly, the tabs 1417 can extend generally perpendicularly to the length, L, of the flex portion 1400 and/or can extend from either side and/or from multiple sides of the flex portion 1400. When the tabs 1417 are at least partially detached from the rings 1418, the rings 1418 can be configured to close a distance previously filled by the tabs 1417 and/or can be configured to move closer together and/or to couple together, as shown in FIG. 14C. With the rings 1418 coupled together and/or situated in close proximity to each other, the flex portion 1400 can have a more rigid structure. In some implementations, one or more pull wires can be used to pull one or more distal rings 1418 to cause the one or more rings 1418 to move together.

The tabs 1417 can connect to the rings 1418 in any suitable manner. For example, at least one side of a tab 1417 can form a releasable and/or detachable connection to a ring 1418. A releasable and/or detachable connection can be formed by a protrusion (e.g., a peg, hook, button, and/or latch) extending from a tab 1417 and/or ring 1418 configured to mate with an aperture, notch, hook, and/or other feature at a ring 1418 and/or tab 1417. In some implementations, a pull wire and/or other device can be used to facilitate detachment of the tabs 1417 from the rings 1418. In some implementations, one or more tabs 1417 can form a detachable connection to a first ring 1418 and/or can form a secure connection to a second ring 1418. For example, as shown in FIG. 14C, the one or more tabs 1417 can remain coupled to the one or more rings 1418 on a second side 1428 while detaching from the one or more rings 1418 at a first side 1427.

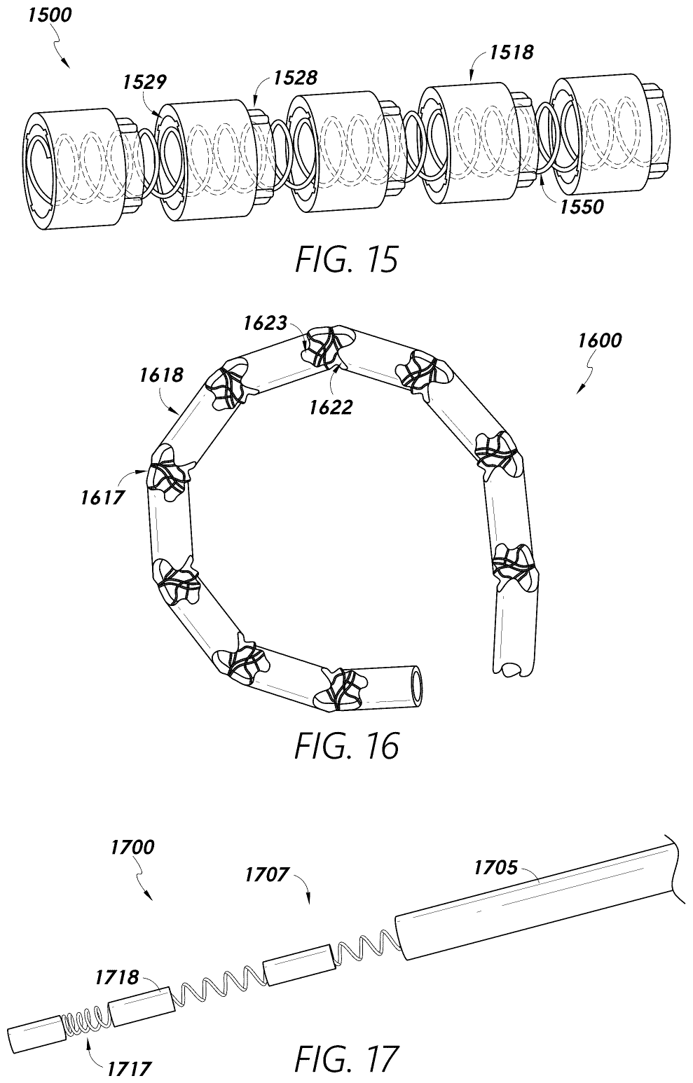

FIG. 15 illustrates an example flex portion 1500 comprising one or more rings 1518 and/or one or more springs 1550 joining the one or more rings 1518, in accordance with one or more implementations. In some implementations, a delivery assembly can comprise the flex portion 1500 and/or a shaft having a generally tubular form and/or comprising an inner lumen. The inner lumen can be configured to receive and/or facilitate conveyance of one or more wires and/or tissue anchors. The flex portion 1500 can similarly have a generally tubular form and/or can comprise an inner lumen. The flex portion 1500 can be a portion/component of an anchor delivery system (e.g., of any of the anchor delivery systems described herein).

The flex portion 1500 can comprise a network of one or more rings 1518 interconnected by one or more springs 1550 and/or coils. The one or more springs 1550 can be configured to extend between multiple rings 1518 of the flex portion 1500. In some implementations, the rings 1518 can have a circular and/or cylindrical form.

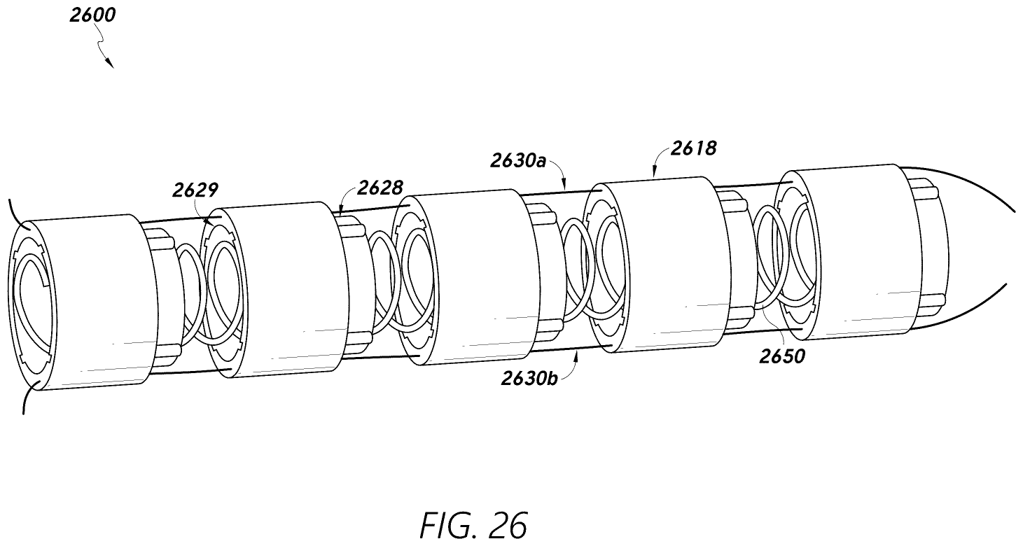

In some implementations, one or more rings 1518 can comprise protrusions 1528 configured to mate with corresponding notches 1529 and/or apertures of another ring 1518. Accordingly, the one or more rings 1518 can be configured to join together. For example, as the one or more rings 1518 are pressed together (e.g., using one or more pull wires configured to apply pulling and/or pushing force to one or more rings 1518), the one or more protrusions 1528 can be configured to slide into the one or more notches and/or can form a secure attachment. While no pull wires are shown in FIG. 15, one or more pull wires can extend through lumens and/or through walls of the rings 1518 (see, e.g., FIG. 26 herein).

The flex portion 1500 can be configured to selectively move between a first state (e.g., an expanded and/or generally flexible state) and a second state (e.g., a compressed and/or generally rigid state). An expanded and/or flexible state is shown in FIG. 15. In the expanded state, there can be at least partial separation between the rings 1518 of the flex portion 1500. The one or more springs 1550 can be configured to interconnect the rings 1518 in the expanded state. Following delivery through narrow anatomical pathways and/or upon arriving at a target tissue site, the rings 1518 can be brought together by any suitable means to move the flex portion 1500 to the compressed state. In the compressed state, the one or more springs 1550 can be at least partially compressed within the rings 1518.

In some implementations, the flex portion 1500 can comprise a single spring 1550 extending at least partially along a length of the flex portion 1500. However, the flex portion 1500 can comprise any number of springs 1550. For example, the flex portion 1500 can comprise a spring 1550 for every pair of rings 1518.

FIG. 16 illustrates an example flex portion 1600 comprising one or more rings 1618 and/or one or more cords 1617 and/or networks of cords 1617 joining the one or more rings 1618, in accordance with one or more implementations. In some implementations, a delivery assembly can comprise the flex portion 1600 and/or a shaft having a generally tubular form and/or comprising an inner lumen. The inner lumen can be configured to receive and/or facilitate conveyance of one or more wires and/or tissue anchors. The flex portion 1600 can similarly have a generally tubular form and/or can comprise an inner lumen. The flex portion 1600 can be a portion/component of an anchor delivery system (e.g., of any of the anchor delivery systems described herein).

The flex portion 1600 can comprise a network of one or more rings 1618 interconnected by one or more cords 1617 and/or cord bundles. The one or more cords 1617 can be configured to extend between adjacent rings 1618 of the flex portion 1600. In some implementations, the rings 1618 can have a circular and/or cylindrical form. For example, the rings 1618 can comprise tubular pieces of material.

The flex portion 1600 can be configured to selectively move between a first state (e.g., an expanded and/or generally flexible state) and a second state (e.g., a compressed and/or generally rigid state). An expanded and/or flexible state is shown in FIG. 16. In the expanded state, there can be at least partial separation between the rings 1618 of the flex portion 1600. The one or more cords 1617 can be configured to interconnect the rings 1618 in the expanded state. Following delivery through narrow anatomical pathways and/or upon arriving at a target tissue site, the rings 1618 can be brought together by any suitable means to move the flex portion 1600 to the compressed state. In the compressed state, the one or more cords 1617 can be situated at least partially within the rings 1618.

In some implementations, a single cord 1617 and/or network/bundle of cords 1617 can be used. In some implementations, multiple distinct cords 1617 and/or cord bundles can be used to interconnect each pair of rings 1618.

The one or more rings 1618 can comprise one or more protrusions 1622 configured to fit into and/or mate with corresponding recesses 1623, notches, and/or apertures of other rings 1618. For example, following delivery of the flex portion 1600 to a target location, force can be applied to the one or more rings 1618 to cause the one or more rings 1618 to move closer together. The protrusions 1622 can fit into the recesses 1623 (e.g., similar to puzzle pieces) to allow the rings 1618 to move closer together and/or to form a more secure connection between the rings 1618 in the rigid state.

FIG. 17 illustrates at least a portion of an assembly 1700 comprising an example flex portion 1707 comprising one or more rings 1718 and/or one or more springs 1717 joining the one or more rings 1718, in accordance with one or more implementations. In some implementations, a delivery assembly can comprise the flex portion 1707 and/or a shaft 1705 having a generally tubular form and/or comprising an inner lumen. The inner lumen can be configured to receive and/or facilitate conveyance of one or more wires and/or tissue anchors. The flex portion 1707 can similarly have a generally tubular form and/or can comprise an inner lumen. The assembly 1700 can be configured as an anchor delivery system or a portion/component of an anchor delivery system (e.g., any of the anchor delivery systems described herein).

The flex portion 1707 can comprise a network of one or more rings 1718 interconnected by one or more springs 1717 and/or coils. The one or more springs 1717 can be configured to extend between multiple rings 1718 of the flex portion 1707. In some implementations, the rings 1718 can have a circular and/or cylindrical form. For example, the rings 1718 can comprise tubular pieces of material.

The flex portion 1707 can be configured to selectively move between a first state (e.g., an expanded and/or generally flexible state) and a second state (e.g., a compressed and/or generally rigid state). An expanded and/or flexible state is shown in FIG. 17. In the expanded state, there can be at least partial separation between the rings 1718 of the flex portion 1707. The one or more springs 1717 can be configured to interconnect the rings 1718 in the expanded state. Following delivery through narrow anatomical pathways and/or upon arriving at a target tissue site, the rings 1718 can be brought together by any suitable means to move the flex portion 1707 to the compressed state. In the compressed state, the one or more springs 1717 can be at least partially compressed within the rings 1718.

In some implementations, the flex portion 1707 can comprise a single spring 1717 extending at least partially along a length of the flex portion 1707. However, the flex portion 1707 can comprise any number of springs 1717. For example, the flex portion 1707 can comprise a spring 1717 between each pair of rings 1718.

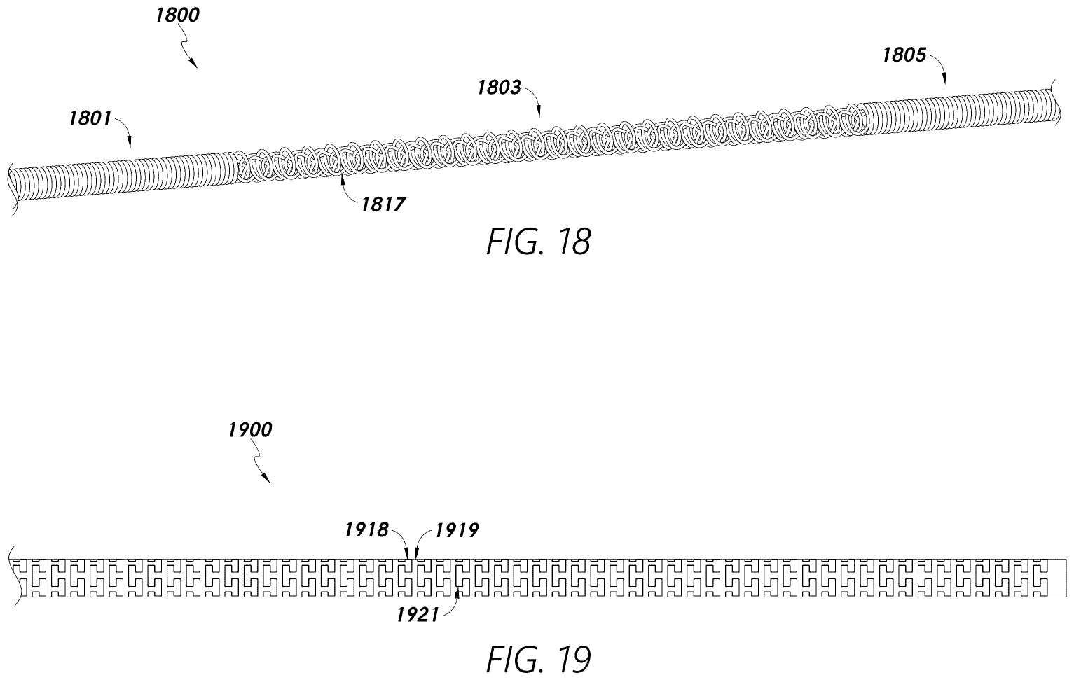

FIG. 18 illustrates at least a portion of an assembly comprising an example flex portion 1800 comprising one or more segments including a first segment 1801, a second segment 1803, and/or a third segment 1805, in accordance with one or more implementations. The second segment 1803 can be situated at least partially between the first segment 1801 and the third segment 1805 and/or can comprise one or more cords 1817 which can be threaded together. The second segment 1803 can be configured to join the first segment 1801 and the third segment 1805. In some implementations, a delivery assembly can comprise the flex portion 1800 and/or a shaft having a generally tubular form and/or comprising an inner lumen. The inner lumen can be configured to receive and/or facilitate conveyance of one or more wires and/or tissue anchors. The flex portion 1800 can similarly have a generally tubular form and/or can comprise an inner lumen. In some implementations, the flex portion 1800 can be a midportion and/or end portion of the shaft. The flex portion 1800 can be a portion/component of an anchor delivery system (e.g., of any of the anchor delivery systems described herein).

The first segment 1801 and/or the third segment 1805 can comprise one or more coiled wires. In some implementations, the second segment 1803 can comprise one or more cords forming the first segment 1801 and/or the third segment 1805 and/or can comprise different cords.

In some implementations, the second segment 1803 can have increased flexibility with respect to the first segment 1801, third segment 1805, and/or other portions of the shaft and/or delivery assembly. The second segment 1803 can be configured to move from a relatively flexible state to a relatively rigid state. For example, by pressing the third segment 1805 towards the first segment 1801 and/or the first segment 1801 towards the third segment 1805, the second segment 1803 can be compressed to assume a more rigid state.

FIG. 19 illustrates an example flex portion 1900 comprising one or more interlocking teeth 1921 in accordance with one or more implementations. In some implementations, a delivery assembly can comprise the flex portion 1900 and/or a shaft having a generally tubular form and/or comprising an inner lumen. The inner lumen can be configured to receive and/or facilitate conveyance of one or more wires and/or tissue anchors. The flex portion 1900 can similarly have a generally tubular form and/or can comprise an inner lumen. The flex portion 1900 can be a portion/component of an anchor delivery system (e.g., of any of the anchor delivery systems described herein).

The flex portion 1900 can comprise any number of teeth 1921 and/or the teeth 1921 can have any suitable shape and/or size. In some implementations, the teeth 1921 can be configured to fit together in a puzzle-piece manner. For example, a first segment 1918 can comprise one or more teeth 1921 configured to mate with corresponding teeth 1921 of a second segment 1919. As shown in FIG. 19, the teeth 1921 can comprise T-shaped protrusions from each segment. However, the teeth 1921 can have other shapes. In some implementations, the first segment 1918 and the second segment 1919 can have generally equal sizes and/or shapes that can be staggered such that protrusions at the first segment 1918 fit into recesses of the second segment 1919 and/or vice versa. The teeth 1921 of one or more segments can continue around the entire circumference of the flex portion 1900.

The flex portion 1900 can be configured to selectively move between a first state (e.g., an expanded and/or generally flexible state) and a second state (e.g., a compressed and/or generally rigid state). In the expanded state, there can be at least partial separation between the teeth 1921 of adjacent segments of the flex portion 1500. Following delivery through narrow anatomical pathways and/or upon arriving at a target tissue site, the teeth 1921 can be brought together by any suitable means to move the flex portion 1900 to the compressed state. In the compressed state, the one or more teeth 1921 can be locked together and/or can be more closely joined than in the expanded state. In some implementations, the teeth 1921 can comprise locking features (e.g., pegs and/or notches) configured to provide a secure attachment between the teeth 1921.

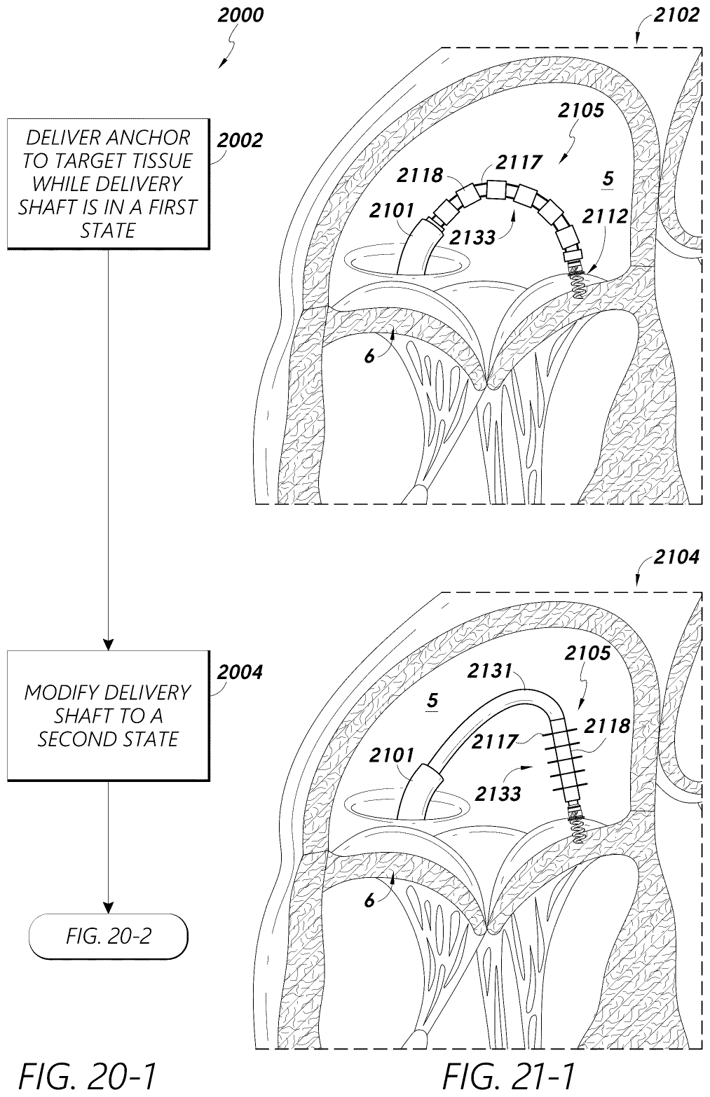

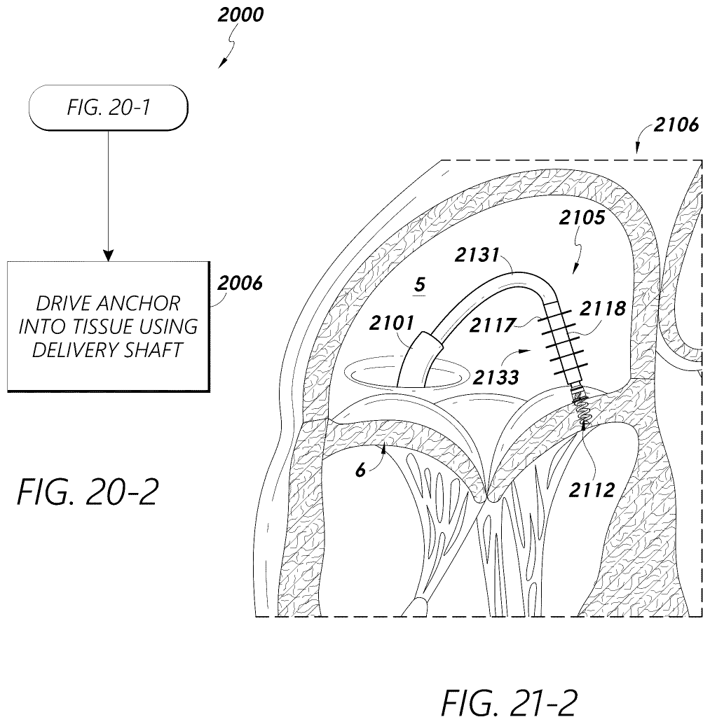

FIG. 20 (FIGS. 20-1 and 20-2) is a flowchart illustrating steps of an example method or process 2000 for delivering one or more tissue anchors via a shaft comprising a flex portion in accordance with one or more implementations. FIG. 21 (FIGS. 21-1 and 21-2) provides example images corresponding to steps of the method/process 2000 of FIG. 20.

At step 2002, the process 2000 involves delivering one or more tissue anchors 2112 to a target tissue site (e.g., a tricuspid valve 6) via a delivery shaft 2105 that is in a first state (e.g., an expanded and/or flexible state) during delivery, as shown in image 2102 of FIG. 21. While a tricuspid valve 6 delivery process is illustrated in FIG. 21, steps of the process 2000 may be applicable to delivery at a variety of tissue sites, including the mitral valve. The delivery shaft 2105 can be a portion/component of an anchor delivery system (e.g., any of the anchor delivery systems described herein).

The delivery shaft 2105 can comprise a flex portion 2133 including any of a variety of features for facilitating transitions of the delivery shaft from the first state to a second state (e.g., a compressed and/or rigid state) and/or from the second state to the first state. For example, as shown in image 2102, the flex portion 2133 can comprise one or more rings 2118 interconnected by one or more tabs 2117. However, the flex portion can have any of the variety of features described herein with respect to flex portions. The shaft 2105 and/or flex portion 2133 can be delivered to the right atrium 5 and/or target tissue site via one or more catheters 2101 (e.g., outer shafts).

At step 2004, the process 2000 involves modifying the delivery shaft 2105 and/or the flex portion 2133 from the first state to a second state (e.g., a compressed and/or rigid state). In some implementations, the flex portion 2133 can comprise one or more tabs 2117 configured to disconnect and/or detach at least partially from one or more rings 2118 to allow the one or more rings 2118 to join together and/or move into close proximity to each other, as shown in image 2104 of FIG. 21. Other example flex portions 2133 can include springs, cords, interlocking teeth, and/or other features configured to facilitate transitions of the shaft 2105 from the first state to the second state and/or vice versa.

In the implementation shown in image 2104, the tabs 2117 can each disconnect on one side from the rings 2118 and/or can extend generally perpendicularly and/or axially from the shaft 2105 and/or tissue anchor 2112. As a result, the rings 2118 can be allowed to fill space previously occupied by the tabs 2117. While in the first state shown in image 2102, the tabs 2117 can extend generally in-line with the shaft 2105 and/or can allow for improved flexibility of the shaft 2105. For example, the one or more tabs 2117 can serve as hinges and/or can comprise a flexible structure that can facilitate bending of the shaft 2105. In some implementations, the rings 2118 can comprise a generally rigid and/or solid material and/or can have minimal flexibility. While in the second state illustrated in image 2104, the one or more rings 2118 can be joined together and/or can close gaps between the rings 2118. As a result, the rigidness of the rings 2118 can cause the flex portion 2133 to have a generally rigid structure.

The shaft 2105 can further comprise one or more proximal segments 2131 extending between the flex portion 2133 and a handle and/or other proximal device. The one or more proximal segments 2131 can have a generally tubular structure and/or can have a generally flexible, semi-flexible, and/or rigid structure. In some implementations, at least a portion of the one or more proximal segments 2131 that is adjacent to the flex portion 2133 can be at least partially flexible to allow the shaft 2105 to bend, as shown in image 2104.

At step 2006, the process 2000 involves driving one or more tissue anchors 2112 into the tissue using the delivery shaft 2105 and/or flex portion 2133 while the flex portion 2133 is in the second state, as shown in image 2106 of FIG. 21. In some implementations, an increased rigidness of the shaft 2105 resulting from the flex portion 2133 being in the second state can facilitate driving the tissue anchors 2112 into the tissue. For example, the flex portion 2133 may not bend in response to driving pressure from the shaft 2105 and into the flex portion 2133.

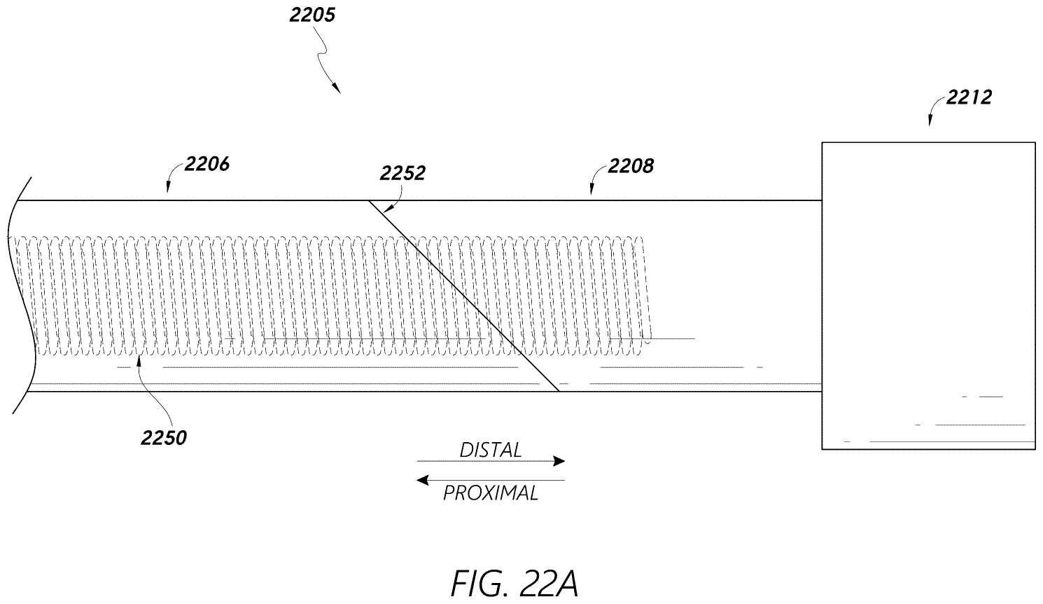

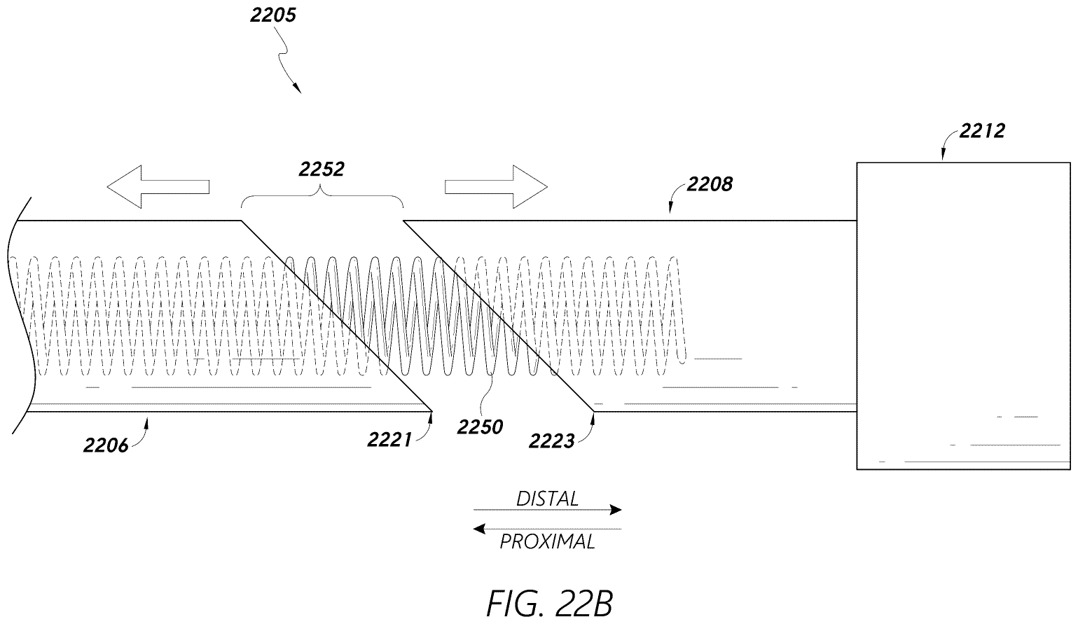

FIGS. 22A and 22B illustrate at least a portion of an example delivery shaft 2205 comprising a flex portion including one or more springs extending across a break in the delivery shaft 2205, in accordance with one or more implementations. In some implementations, the delivery shaft 2205 can comprise a driver 2212 at a distal end of the delivery shaft 2205 configured to drive one or more tissue anchors into target tissue locations (e.g., at or around one or more valves of a heart). The driver 2212 can have an increased width and/or diameter relative to a width and/or diameter of other portions of the delivery shaft 2205. The delivery shaft 2205 can be a portion/component of an anchor delivery system (e.g., any of the anchor delivery systems described herein).

In some implementations, the delivery shaft 2205 can comprise a flex portion at or near the driver 2212 and/or a distal end of the delivery shaft 2205. The flex portion can be configured to facilitate a change from a first state (e.g., a generally linear and/or continuous state) to a second state (e.g., a generally bent and/or discontinuous state) and/or from the second state to the first state.

The flex portion can comprise a break 2252 and/or cut in the delivery shaft 2205. In some implementations, the break 2252 can comprise a cut through the shaft 2205 that separates the shaft into a proximal segment 2206 and a distal segment 2208. While the break 2252 is shown as a diagonal cut through the shaft 2205 in FIGS. 22A and 22B, the break 2252 can be any type of cut and/or can have any angle(s).

In some implementations, the flex portion can comprise one or more springs 2250 and/or similar devices extending across the break 2252 and/or situated at least partially within the proximal segment 2206 and the distal segment 2208. The one or more springs 2250 can be configured to form a bridge between the proximal segment 2206 and the distal segment 2208 and/or to press the proximal segment 2206 and the distal segment 2208 into contact with each other. The one or more springs 2250 can be configured to assume a resting, relaxed, and/or compressed form when the proximal segment 2206 and the distal segment 2208 are in a continuous and/or linear alignment, as shown in FIG. 22A.

As shown in FIG. 22B, the one or more springs 2250 can be configured to expand and/or twist as necessary to maintain a connection between the proximal segment 2206 and the distal segment 2208 even when the proximal segment 2206 and the distal segment 2208 are not in contact with each other and/or are not in alignment. If the break 2252 between the proximal segment 2206 and the distal segment 2208 widens, the one or more springs 2250 can assume an expanded and/or stretched form to extend across any gap between the proximal segment 2206 and the distal segment 2208.

In some implementations, the shaft 2205 can have a default and/or natural alignment of the proximal segment 2206 to the distal segment 2208. For example, a proximal edge 2221 of the proximal segment 2206 can be configured to be aligned with a distal edge 2223 of the distal segment 2208 and/or the proximal segment 2206 and the distal segment 2208 can naturally be in contact. In the first state, the proximal edge 2221 and the distal edge 2223 can be in contact and/or in close proximity to each other. The one or more springs 2250 can be configured to maintain alignment of the proximal segment 2206 with the distal segment 2208. Accordingly, the one or more springs 2250 can be configured to resist misalignment of the proximal segment 2206 and the distal segment 2208. For example, if a twisting force is applied to the proximal segment 2206, the one or more springs 2250 can be configured to maintain alignment and/or to resist the proximal segment 2206 from twisting out of alignment with the distal segment 2208. If the proximal segment 2206 and/or distal segment 2208 is twisted out of alignment, the one or more springs 2250 can be configured to elastically deform as necessary to maintain a bridge between the proximal segment 2206 and the distal segment 2208 while providing a counter force to pull the proximal segment 2206 and the distal segment 2208 back into alignment.

The flex portion can be configured to provide torque limiting at the distal end and/or distal portion 2208 of the shaft 2205. For example, the flex portion can be configured to provide a measure and/or read on when an amount of torque at the distal portion 2208 of the shaft 2205 exceeds a given amount. The flex portion can be designed to become misaligned when the distal portion 2208 of the shaft 2205 reaches a desired and/or set maximum torque level.

While the shaft 2305 is shown comprising an angled break 2252 (e.g., an approximately 45-degree break and/or cut), the break 2252 can have a different angle and/or can be generally perpendicular. The one or more coils 2250 and/or springs can be configured to maintain alignment of the proximal segment 2206 and the distal segment 2208 while the shaft 2205 is twisted until the twisting causes the torque of the distal segment 2208 to exceed a predetermined level. For example, the one or more coils 2250 can be configured to transfer torque from the proximal segment 2206 to the distal segment 2208. In some implementations, the proximal segment 2206 and/or distal segment 2208 can additionally or alternatively comprise various features to increase friction and/or attachment between the proximal segment 2206 and the distal segment 2208 to facilitate transfer of torque from the proximal segment 2206 to the distal segment 2208. For example, the proximal segment 2206 and/or distal segment 2208 can comprise one or more pegs, bumps, and/or other protrusions configured to mate with corresponding notches and/or indentations at the distal segment 2208 and/or proximal segment 2206.

The flex portion can be configured to facilitate various methods of testing how well a tissue anchor is anchored into tissue. For example, after the shaft 2205 has been twisted at least somewhat to cause a tissue anchor at a distal end of the shaft 2205 to be at least partially embedded in tissue, a surgeon may axially pull back on the shaft 2205 and/or on the proximal segment 2206 of the shaft 2205. The amount of resistance presented by the distal segment 2208 can indicate how well the tissue anchor is embedded and/or can be reflected by how easily the proximal segment 2206 is pulled away from the distal segment 2208 to create a gap 2252 between the proximal segment 2206 and the distal segment 2208. For example, if the distal segment 2208 remains in place while the proximal segment 2206 is pulled away from the distal segment 2208 and/or a predetermined amount of separation is achieved before the distal segment 2208 is pulled out, the tissue anchor may be suitably embedded and may not require further anchoring. In contrast, if the distal segment 2208 pulls away from the tissue with the proximal segment 2206 and/or does not provide a suitable amount of resistance when the proximal segment 2206 is pulled, further anchoring may be required.

In some implementations, the break 2252 can be configured to allow at least partial separation between the proximal segment 2206 and the distal segment 2208 during delivery of the shaft 2205 to a target tissue location. For example, as the shaft 2205 moves around bends in the patient's anatomy, the break 2252 can widen to allow increased bending of the shaft 2205. The shaft 2205 can be at least partially composed of braided cable to allow for some flexibility of the shaft 2205. Moreover, a thickness of the shaft 2205 can be variable across the shaft 2205 as needed to facilitate delivery of the shaft 2205. For example, the shaft 2205 can have reduced thickness at or near the flex portion and/or distal portion of the shaft 2205 to facilitate bending of the flex portion and/or distal portion.

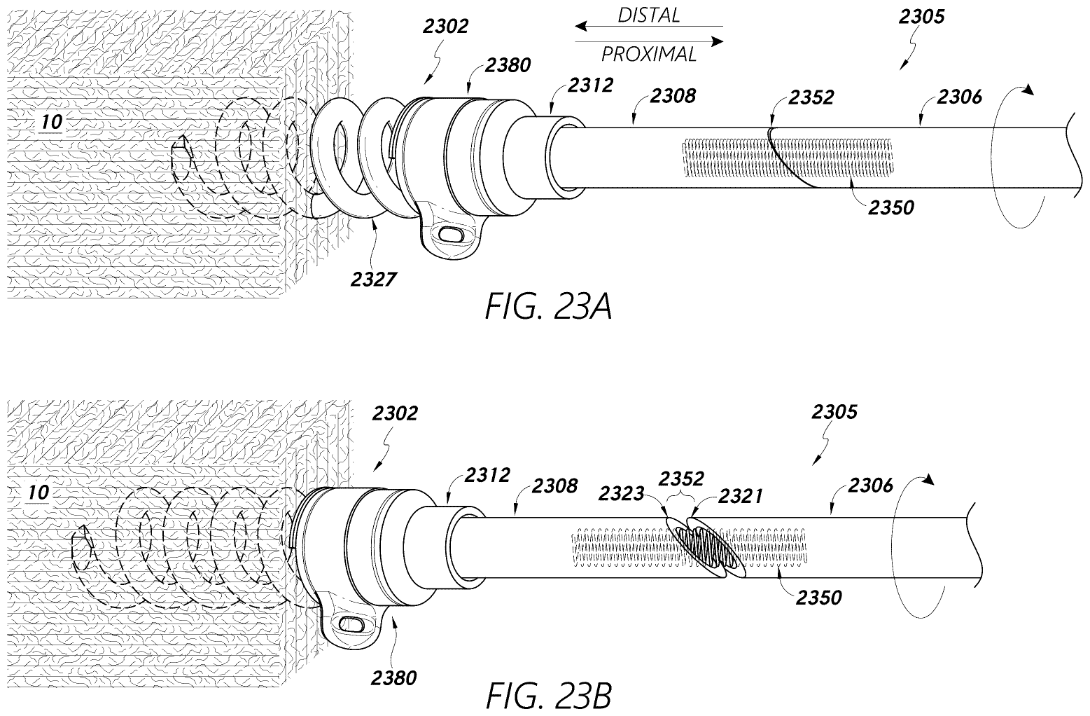

FIGS. 23A and 23B illustrate driving process for one or more tissue anchors 2302 into tissue 10 in accordance with one or more implementations. In some implementations, a shaft 2305 can comprise a driver 2312 configured to interface with a tissue anchor 2302 (e.g., at a head 2380 portion of the anchor 2302) to twist and/or press the tissue anchor 2302 into the tissue. The shaft 2305 can comprise a flex portion comprising one or more springs 2350 bridging a break 2352 in the shaft 2305. The break 2352 can represent a separation between a proximal segment 2306 and a distal segment 2308 of the shaft 2305. During delivery and/or before a tissue anchor at the distal end of the shaft 2305 is fully embedded, contact between the proximal segment 2306 and the distal segment 2308 can be flush. The driver 2312 and/or the shaft 2305 and associated features can be configured as an anchor delivery system (which can be similar to other anchor delivery systems herein) or a portion/component of an anchor delivery system.

In some implementations, the shaft 2305 can be configured to be twisted to cause corresponding twisting of the driver 2312 and/or tissue anchor 2302. In some implementations, the tissue anchor 2302 can comprise a coil 2327 configured to embed into the tissue 10 as it is twisted. Thus, as the shaft 2305 is twisted, the tissue anchor 2302 can embed further into the tissue 10 as shown in FIG. 23B.

When at least a portion of the tissue anchor 2302 (e.g., a coil of the tissue anchor 2302) is fully embedded in the tissue 10, a base portion of the tissue anchor 2302 can press against the tissue 10 and/or the tissue anchor 2302 can provide an increased amount of resistance to the driver 2312 and/or the shaft 2305. This increased resistance can cause the distal segment 2308 of the shaft 2305 to resist further twisting.

The proximal segment 2306 can be situated between the distal segment 2308 and a handle and/or other control mechanism. Force from the handle and/or other control mechanism can be translated first to the proximal segment 2306 and then to the distal segment 2308 via the one or more springs 2350 and/or friction between the proximal segment 2306 and the distal segment 2308. When the distal segment 2308 presents increased resistance as a result of the tissue anchor 2302 being fully and/or at least partially embedded in the tissue 10, twisting of the proximal segment 2306 may not translate to twisting of the distal segment 2308. As a result, the proximal segment 2306 may twist out of alignment with the distal segment 2308, as shown in FIG. 23B.

When the proximal segment 2306 twists out of alignment with the distal segment 2308, the break 2352 between the proximal segment 2306 and the distal segment 2308 can at least partially widen and/or one or more gaps can form between the proximal segment 2306 and the distal segment 2308. Moreover, a proximal edge 2321 of the proximal segment 2306 can move out of contact and/or out of alignment with a distal edge 2323 of the distal segment 2308.

In some implementations, the flex portion of the shaft 2305 can be at least partially viewable to surgeons via any suitable scope devices and/or similar methods. Accordingly, when the proximal segment 2306 and the distal segment 2308 move out of alignment, the misalignment can be visible and/or noticeable to surgeons. Using this information, a surgeon and/or driving mechanism can discontinue applying driving force and/or can decouple the shaft 2305 and/or driver 2312 from the tissue anchor 2302. In some implementations, the flex portion can be configured to serve as a slip clutch and/or can be configured to prevent more than a configured threshold torque from being applied.

Various factors determining how much torque can be applied before the shaft 2305 reaches distortion and/or becomes misaligned can include a strength of the coil 2350 and/or surface characteristics (e.g., frictional coefficients) between the proximal segment 2306 and the distal segment 2308. The shaft 2305 can be calibrated based on how much torque may be needed to drive a tissue anchor into the tissue. The flex portion can be configured to limit the forces applied in the torque direction and/or in the push/pull and/or linear direction.

A slanted and/or angled break 2352 can be configured to facilitate testing of torque and/or linear directions. For example, the break 2352 can be configured to cause distortion as a result of too much torque and/or pulling force. Separation of the proximal segment 2306 and the distal segment 2308 can be identified in any suitable manner, which can include fluoroscopically and/or electrically. In some implementations, the break 2352 can be oblique and/or helical.

Friction along the length of the shaft 2305 may depend and/or may be determined at least in part based on the curvature of the catheter(s) around the shaft 2305. For example, the more curvature of a catheter around the shaft 2305, the more friction at the shaft 2305. In some cases, a catheter around the shaft 2305 can constrain the shaft 2305 to some extent and/or can help prevent and/or inhibit the shaft 2305 from whipping and/or otherwise moving out of alignment and/or may hold the shaft 2305 in position, which can build up friction at the shaft 2305. Wherever an outer catheter resists movement of the shaft 2305, some torque at the shaft 2305 may be lost.

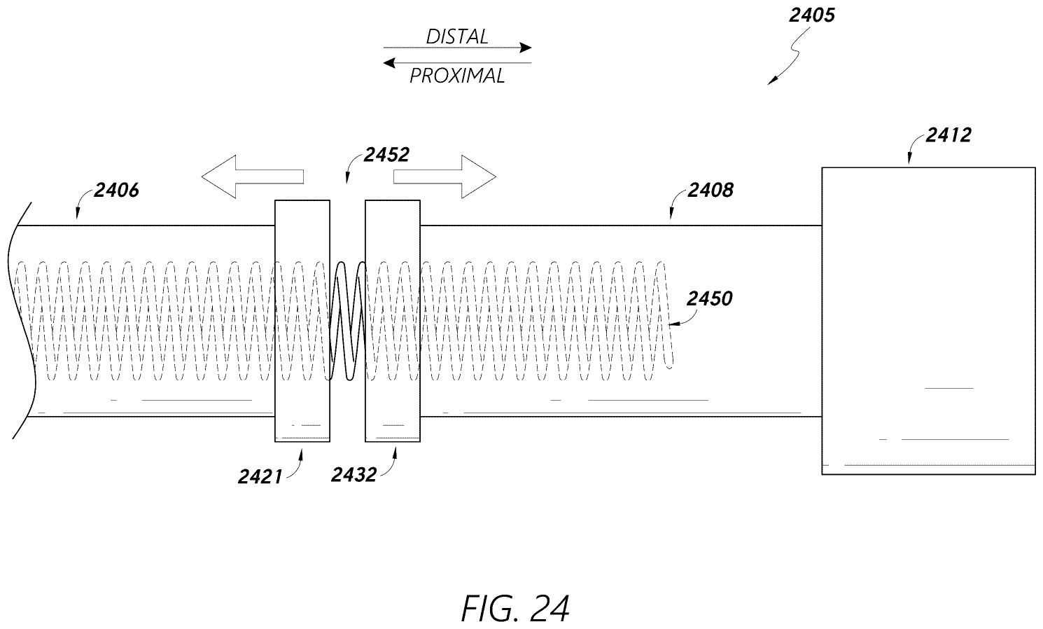

FIG. 24 illustrates an example shaft 2405 comprising a flex portion in accordance with one or more implementations. The shaft 2405 can be a component/portion of an anchor delivery system (e.g., similar to other anchor delivery systems herein). The flex portion can comprise one or more coils 2450 situated at least partially within the shaft 2405 and/or configured to extend between a distal segment 2408 and a proximal segment 2406 of the shaft 2405. The flex portion can further comprise a break 2452 and/or gap between the proximal segment 2406 and the distal segment 2408. The break 2452 can be a generally perpendicular and/or 90-degree cut in the shaft 2405, as illustrated in FIG. 24. However, the break 2452 can comprise a diagonal cut and/or can have any suitable angle.

In some implementations, the shaft 2405 and/or flex portion can comprise one or more edge portions, which can protrude from the shaft 2405. For example, the proximal segment 2406 can comprise a first edge portion 2421 and/or the distal segment 2408 can comprise a second edge portion 2432. The first edge portion 2421 and/or the second edge portion 2432 can be situated at either side of the break 2452 and/or can be configured to be in contact with each other at a resting state of the shaft 2405. The shaft 2405 is illustrated in FIG. 24 with a gap formed between the proximal segment 2406 and the distal segment 2408 for illustrative purposes. However, the one or more coils 2450 and/or springs can be configured to pull the proximal segment 2406 and the distal segment together 2408 such that the first edge portion 2421 and the second edge portion 2432 are in contact at a resting and/or default state.

The shaft 2405 can be configured to be twisted to facilitate driving one or more tissue anchors into tissue via a driver 2412 at a distal end of the shaft 2405. Based at least in part on the break 2452 between the proximal segment 2406 and the distal segment 2408, the proximal segment 2406 can be configured to twist out of alignment with the distal segment 2408 when at least a portion of the tissue anchor is fully embedded in the tissue.

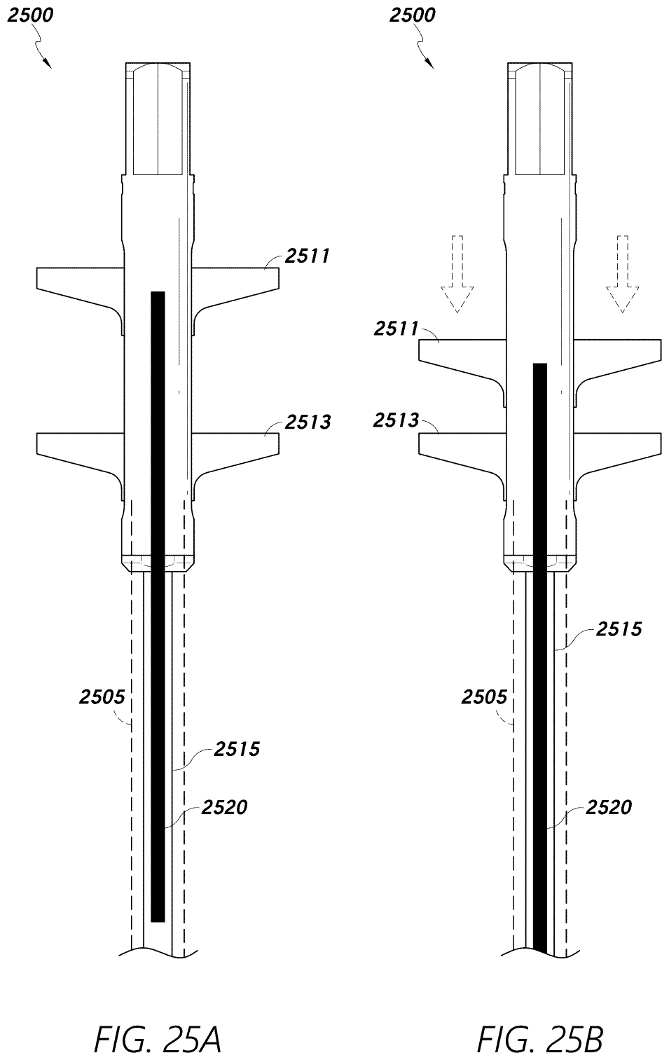

FIGS. 25A and 25B illustrate an example delivery device 2500 for delivering one or more catheters and/or medical devices to a target location within a body, in accordance with one or more implementations. The delivery device 2500 can be configured as an anchor delivery system (which can be similar to other anchor delivery systems herein) or a portion/component of an anchor delivery system. The delivery device 2500 can comprise multiple handles and/or engagement mechanisms, including a first handle 2511 and/or a second handle 2513. The first handle 2511 can be configured to extend and/or control extension of a wire 2520 (e.g., a Nitinol wire) and/or medical implant through an outer catheter 2505 and/or an inner catheter 2515. The second handle 2513 can be configured to extend and/or control extension of one or more pull wires. One or more pull wires can be situated at least partially within the outer catheter 2505 and/or inner catheter 2515 and/or can be configured to facilitate movement of one or more flex portions at the outer catheter 2505 and/or inner catheter 2515 from an expanded and/or flexible state to a compressed and/or rigid state. FIG. 25A illustrates the device 2500 in a first state in which the first handle 2511 has not been engaged to cause extension and/or protrusion of the wire 2520. FIG. 25B illustrates a second state of the device 2500 in which the first handle 2511 has been pressed towards the second handle 2513 to cause extension and/or protrusion of the wire 2520 with respect to the outer catheter 2505 and/or inner catheter 2515.

FIG. 26 illustrates an example flex portion 2600 comprising one or more pull wires 2630 configured to control positioning of one or more rings 2618 and/or one or more springs 2650 joining the one or more rings 2618, in accordance with one or more implementations. In some implementations, a delivery assembly can comprise the flex portion 2600 and/or a shaft having a generally tubular form and/or comprising an inner lumen. The inner lumen can be configured to receive and/or facilitate conveyance of one or more wires and/or tissue anchors. The flex portion 2600 can similarly have a generally tubular form and/or can comprise an inner lumen. The flex portion 2600 can be a portion/component of an anchor delivery system (which can be the same as or similar to other anchor delivery systems herein).

The flex portion 2600 can comprise a network of one or more rings 2618 interconnected by one or more springs 2650 and/or coils. The one or more springs 2650 can be configured to extend between multiple rings 2618 of the flex portion 2600. In some implementations, the rings 2618 can have a circular and/or cylindrical form.

In some implementations, one or more rings 2618 can comprise protrusions 2628 configured to mate with corresponding notches 2629 and/or apertures of another ring 2618. Accordingly, the one or more rings 2618 can be configured to join together.

The flex portion 2600 can be configured to selectively move between a first state (e.g., an expanded and/or generally flexible state) and a second state (e.g., a compressed and/or generally rigid state). An expanded and/or flexible state is shown in FIG. 26. In the expanded state, there can be at least partial separation between the rings 2618 of the flex portion 2600. The one or more springs 2650 can be configured to interconnect the rings 2618 in the expanded state. Following delivery through narrow anatomical pathways and/or upon arriving at a target tissue site, the rings 2618 can be brought together by any suitable means to move the flex portion to the compressed state. In the compressed state, the one or more springs 2650 can be at least partially compressed within the rings 2618.

In some implementations, the flex portion 2600 can comprise a single spring 2650 extending at least partially along a length of the flex portion 2600. However, the flex portion 2600 can comprise any number of springs 2650. For example, the flex portion 2600 can comprise a spring 2650 for every pair of rings 2618.

The flex portion 2600 and/or a delivery device delivering the flex portion 2600 can comprise one or more pull wires 2630 configured to extend at least partially through the one or more rings 2618 to control positions of the one or more rings 2618 with respect to each other. For example, the one or more pull wires 2630 can pass through apertures of the rings 2618 and/or can be pulled to pull a distal ring 2618 towards the proximal rings 2618. By pulling the rings 2618 together, the one or more pull wires 2630 can be configured to facilitate movement of the flex portion 2600 from a flexible and/or expanded state to a compressed and/or rigid state.

The flex portion 2600 is shown comprising a first pull wire 2630a and a second pull wire 2630b. However, the flex portion 2600 and/or delivery device can comprise any number of pull wires 2630. For example, the flex portion 2600 and/or delivery device can comprise four pull wires 2630. In some implementations, two pull wires 2630 can be situated at approximately opposing sides of a ring 2618, as shown in FIG. 26. In some implementations, the one or more pull wires 2630 can be configured to extend through each ring 2618 of the flex portion 2600. However, the one or more pull wires 2630 can be configured to extend through only a portion of the flex portion 2600.

In accordance with one or more implementations of the present disclosure, a method for delivering one or more tissue anchors comprises delivering an elongate shaft carrying one or more tissue anchors at a distal end of the elongate shaft to a target tissue location. The elongate shaft comprises a flex portion configured to move between a first state and a second state. The method can further comprise moving the flex portion from the first state to the second state to facilitate driving the one or more tissue anchors into the target tissue location and moving the flex portion from the second state to the first state.

Moving the flex portion from the first state to the second state can involve compressing the flex portion. In some implementations, moving the flex portion from the first state to the second state involves twisting the elongate shaft until a gap forms in the flex portion.

In some implementations, the flex portion comprises at least one detachable tab between segments of the flex portion. The segments can be ring-shaped.

The flex portion can comprise a network of interlocking teeth. In some implementations, the flex portion comprises a coil extending through a lumen of the flex portion.

In some implementations, the coil is configured to interconnect segments of the flex portion. The flex portion can comprise a cut through the elongate shaft.

Moving the flex portion from the first state to the second state involves pulling one or more pull wires extending at least partially through the flex portion.

The above method(s) can be performed on a living animal or on a simulation, such as on a cadaver, cadaver heart, simulator (e.g., with the body parts, heart, tissue, etc. being simulated), etc.

Some implementations of the present disclosure relate to a system for delivering one or more tissue anchors comprising an elongate shaft carrying one or more tissue anchors at a distal end of the elongate shaft to a target tissue location. The elongate shaft comprises a flex portion configured to move between a first state and a second state.

In some implementations, the system further comprises an inner shaft configured to extend at least partially through a lumen of the elongate shaft. The system can further comprise an outer shaft configured to at least partially enclose the elongate shaft.

The flex portion can be configured to move from the first state to the second state by compressing the flex portion. In some implementations, the flex portion is configured to form a gap in response to twisting of the elongate shaft.

In some implementations, the flex portion comprises at least one detachable tab between segments of the flex portion. The segments can be ring-shaped.

The flex portion can comprise a network of interlocking teeth. In some implementations, the flex portion comprises a coil extending through a lumen of the flex portion.

In some implementations, the coil is configured to interconnect segments of the flex portion. The flex portion can comprise a cut through the elongate shaft.

The system can further comprise one or more pull wires configured to extend at least partially through the flex portion. In some implementations, the one or more pull wires extend at least partially through multiple segments of the flex portion.

### Sterilization

Any of the various systems, devices, apparatuses, etc. in this disclosure can be sterilized (e.g., with heat, radiation, ethylene oxide, hydrogen peroxide, etc.) to ensure they are safe for use with patients, and any of the methods herein can include sterilization of the associated system, device, apparatus, etc. (e.g., with heat, radiation, ethylene oxide, hydrogen peroxide, etc.) as one of the steps of the method.

### Compressible Anchor Driver

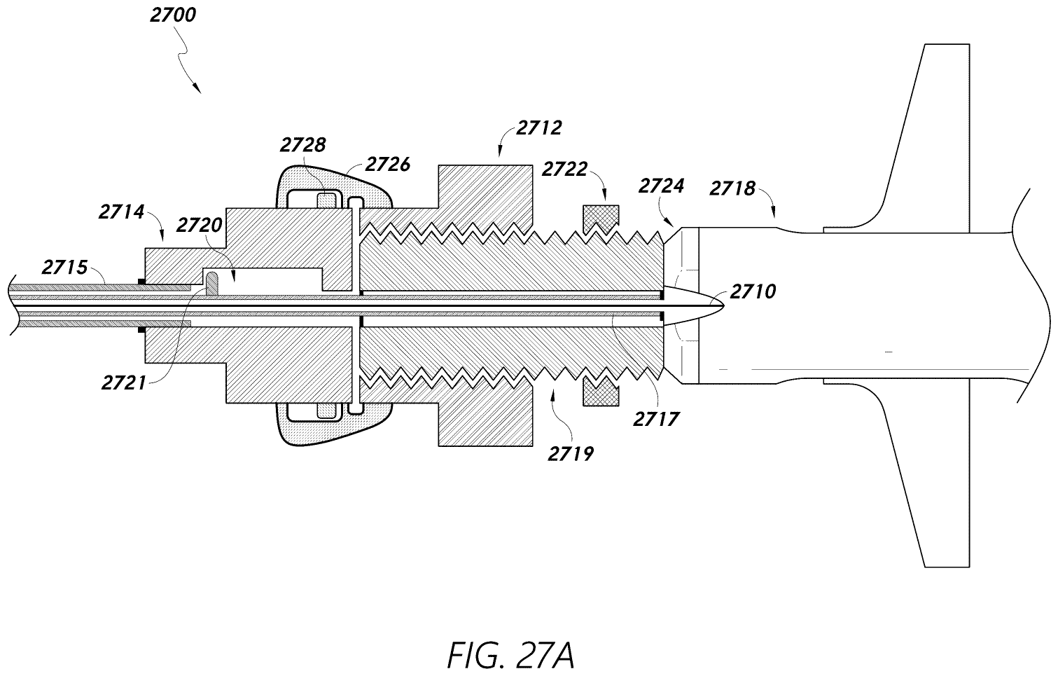

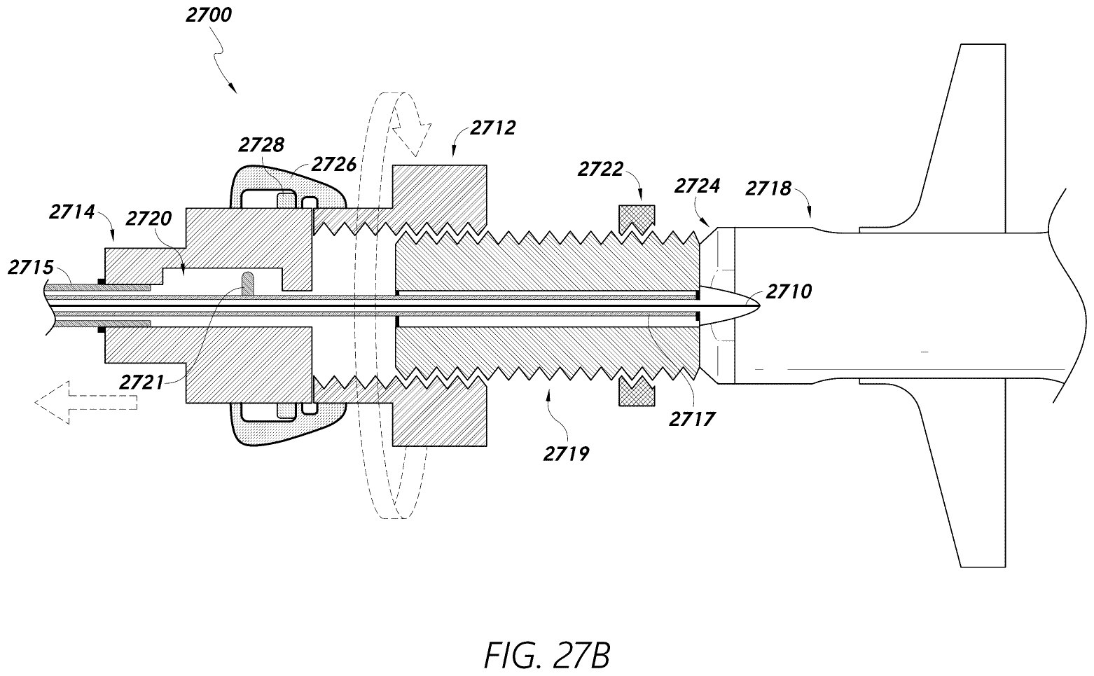

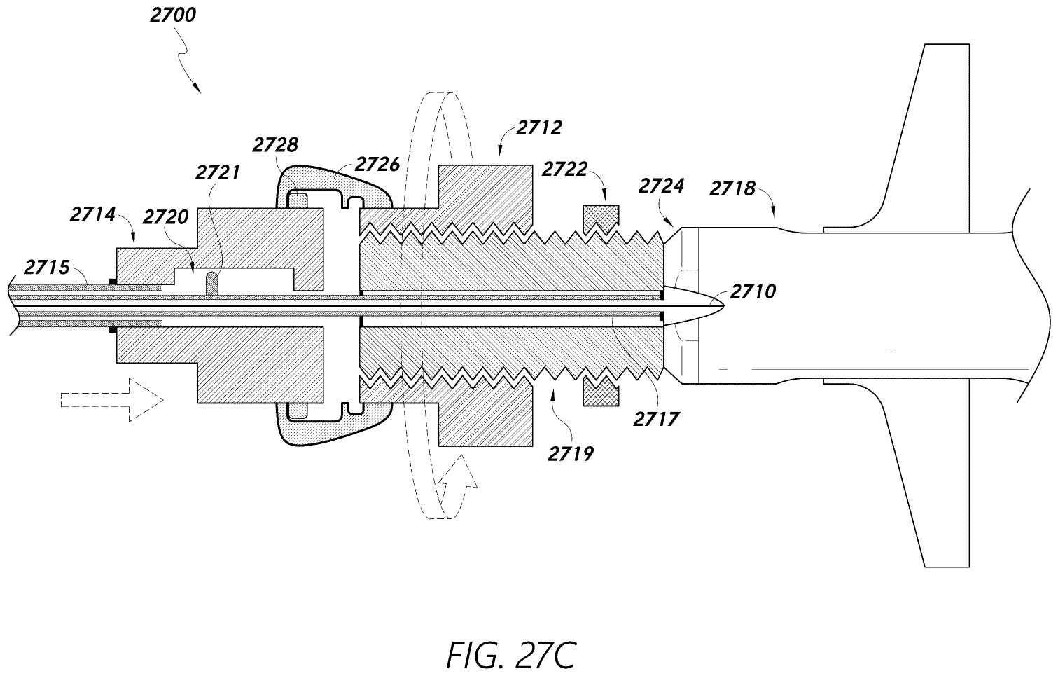

FIGS. 27A-27C illustrate an example anchor delivery system 2700 configured for delivery of one or more tissue anchors in accordance with one or more examples. The anchor delivery system 2700 can be configured to be compressible and/or adjustable. The anchor delivery system 2700 can include one or more shafts and/or other components (e.g., stiffening components, steering components, softening components, flexibility components, etc.) or features. The anchor delivery system can include a portion or component (e.g., outer shaft, inner shaft, rod, wire, extension, etc.) that moves (e.g., axially, rotationally, etc.) relative to another portion or component (e.g., outer shaft, inner shaft, rod, wire, extension, etc.) to change one or more of the stiffness, rigidity, softness, flexibility, etc. of the anchor delivery system.

In some implementations, the system 2700 can be configured to extend an outer shaft 2715 of the system 2700 without twisting the outer shaft 2715. The outer shaft 2715 may not be required to resist torque loads. For example, torque buildup at a distal end of the outer shaft 2715 may cause binding of the outer shaft 2715 and/or one or more anchors interfacing with the distal end of the outer shaft 2715. The system 2700 can additionally comprise an inner shaft 2717. The outer shaft 2715 and/or inner shaft 2717 can be coupled to a gripper (not shown; see, e.g., FIG. 11) at a distal end of the system 2700.

In some implementations, the outer shaft 2715 and/or inner shaft 2717 can be configured to receive one or more wires 2710 (e.g., Nitinol wires). While only a single wire 2710 is shown in FIGS. 27A-27C, the system 2700 can comprise any number of wires 2710. In some implementations, one or more wires 2710 can be configured for moving flex portions of the outer shaft 2715 and/or inner shaft 2717 between multiple states. For example, the one or more wires 2710 can comprise pull wires configured to be selectively tensioned to cause movement of flex portions of the various shafts and/or catheters.

In some implementations, the outer shaft 2715 and/or inner shaft 2717 can comprise laser cut hypo tubes and/or similar components. In some implementations, the outer shaft 2715 and/or inner shaft 2717 can comprise one or more bits and/or similar devices at distal ends of the outer shaft 2715 and/or inner shaft 2717 configured to fit with a gripper at a distal end of the system 2700. Twisting of the outer shaft 2715 and/or inner shaft 2717 may create binding issues at the gripper and/or other area. Such binding issues may result from metal-on-metal contact between a metallic gripper and/or metallic outer shaft 2715 and/or inner shaft 2717. In some implementations, the system 2700 may advantageously not require twisting of the outer shaft 2715 and/or can involve longitudinal extension of the outer shaft 2715 with minimal or no torque on the outer shaft 2715. The system 2700 can further advantageously prevent unnecessary and/or excessive compression of the inner shaft 2717.

In some implementations, the outer shaft 2715 and/or inner shaft 2717 can be welded to the gripper to fix the gripper and/or distal end of the system 2700 in place. In some implementations, the system 2700 can comprise a nut 2712 (e.g., screw, bolt, and/or threaded member) configured to apply force and/or compression to a push knob 2714 and/or to the outer shaft 2715. In some implementations, the outer shaft 2715 can be coupled to the push knob 2714. In some implementations, the nut 2712 can be threaded and/or can be configured to twist along a threaded surface of a set screw 2719 (e.g., threaded screw).

Twisting and/or torque of the nut 2712 may not be translated to the knob 2714 and/or outer shaft 2715. For example, the nut 2712 can be configured to slide along a surface of the knob 2714 to press the knob 2714 without creating corresponding twisting in the knob 2714. In some implementations, the system 2700 can comprise a washer and/or slipping member (e.g., bearing surface, Teflon washer, etc.) between the nut 2712 and the knob 2714 to prevent frictional torque transmission from the nut 2712 to the knob 2714. The nut 2712 may not be attached to the outer shaft 2715. The knob 2714 can be configured to not twist in response to force applied from the nut 2712.

In some implementations, the knob 2714 can comprise a channel 2720 configured to receive a pin 2721 and/or protrusion extending from the inner shaft 2717. The pin 2721 can be configured to prevent rotation and/or extension of the knob 2714. The channel 2720 can have a generally rectangular shape and/or can comprise a recess and/or cavity through the knob 2714. The inner shaft 2717 can be configured to extend at least partially through the knob 2714 and/or the screw 2719.

As shown in FIG. 27B, in some implementations, rotation (e.g., clockwise and/or counterclockwise rotation) of the nut 2712 can cause axial extension of the nut along the set screw 2719. For example, the nut 2712 can be configured to slide and/or glide along threads of the set screw 2719. In some implementations, movement and/or extension of the nut 2712 along the screw 2719 can be configured to cause corresponding movement and/or extension of the knob 2714. For example, the nut 2712 can be configured to press against the knob 2714 and/or to exert an axial and/or pressing force against the knob 2714. The knob 2714 can be configured to not twist in response to force applied by the nut 2712.

In some implementations, the system 2700 can comprise one or more arms 2726 coupled to the nut 2712 and/or knob 2714 and/or configured to form one or more bridges between the nut 2712 and the knob 2714. For example, the knob 2714 can comprise one or more pegs 2728, which can include any knobs, notches and/or other protrusions configured to be grasped and/or otherwise engaged by the one or more arms 2726. In some implementations, the one or more pegs 2728 can comprise an oval shaped extension from the knob 2714 extending around an entire circumferential surface of the knob 2714 and/or can comprise one or more discrete protrusions configured to engage with discrete arms 2726 coupled to the nut 2712. While FIGS. 27A-27C illustrate the pegs 2728 extending from the knob 2714, the knob 2714 can additionally or alternatively comprise one or more cavities, apertures, and/or recesses configured to receive corresponding protrusions from the one or more arms 2726.

In some implementations, the one or more arms 2726 can be configured to apply pushing and/or pulling force to the one or more pegs 2728. For example, as the nut 2712 is extended, the one or more arms 2726 can be configured to apply force to proximal surfaces of the one or more pegs 2728. Similarly, when the nut 2712 is rotated in an opposite direction as illustrated in FIG. 27C, the nut 2712 can be retracted and/or can be configured to apply pulling force to distal surfaces of the one or more pegs 2728. In some implementations, the one or more arms 2726 can comprise proximal and/or distal walls and/or protrusions configured to engage the proximal and/or distal sides of the pegs 2728, respectively. In some implementations, the one or more arms 2726 can comprise hollow spherical extensions configured to surround the one or more pegs 2728.

In some implementations, the system 2700 can comprise a stopper 2722 (e.g., stopper nut) configured to prevent retraction of the nut 2712 beyond a certain point along the screw 2719. For example, the stopper 2722 can be disposed along the screw 2719 between the nut 2712 and the handle 2718. The system 2700 can comprise a handle nut 2724 disposed between the screw 2719 and the handle 2718.

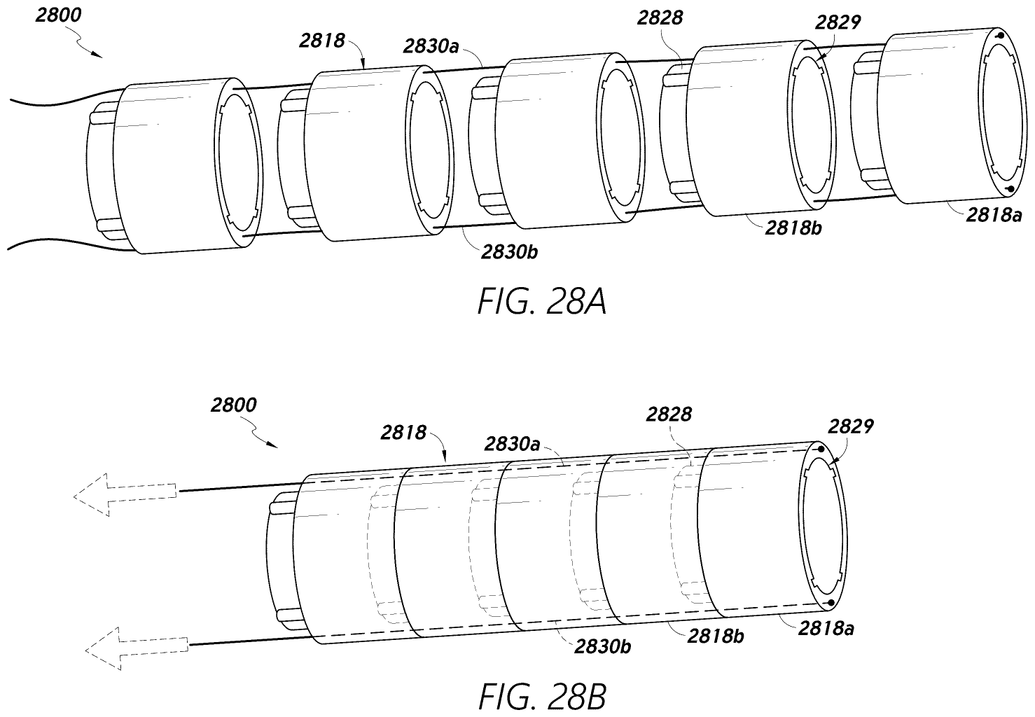

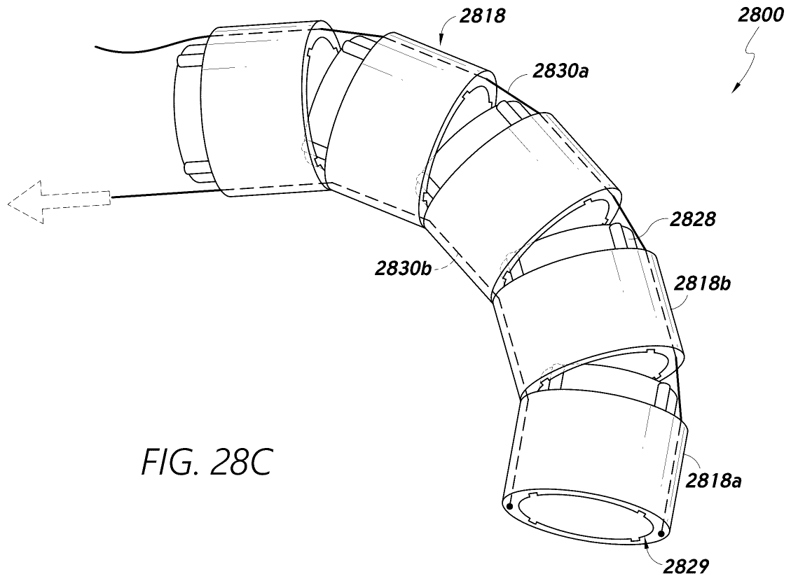

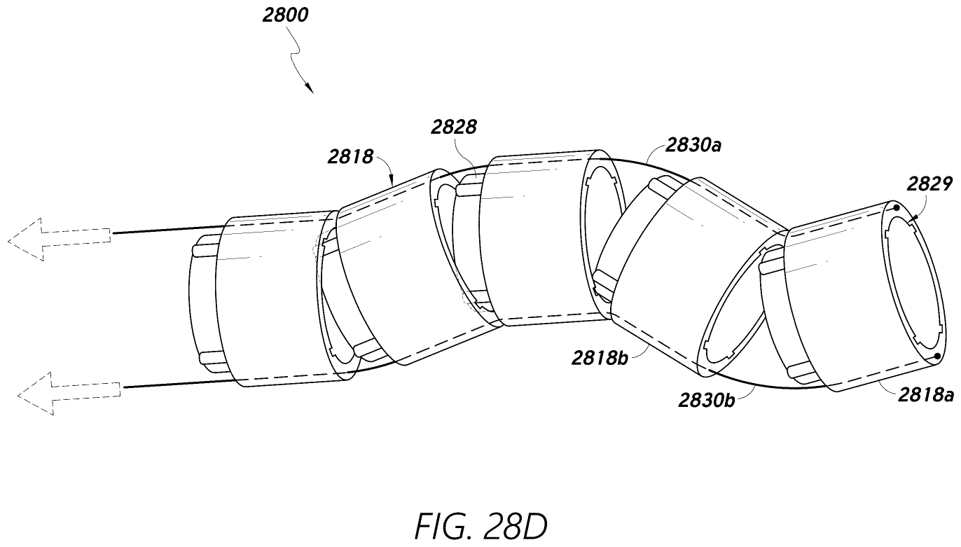

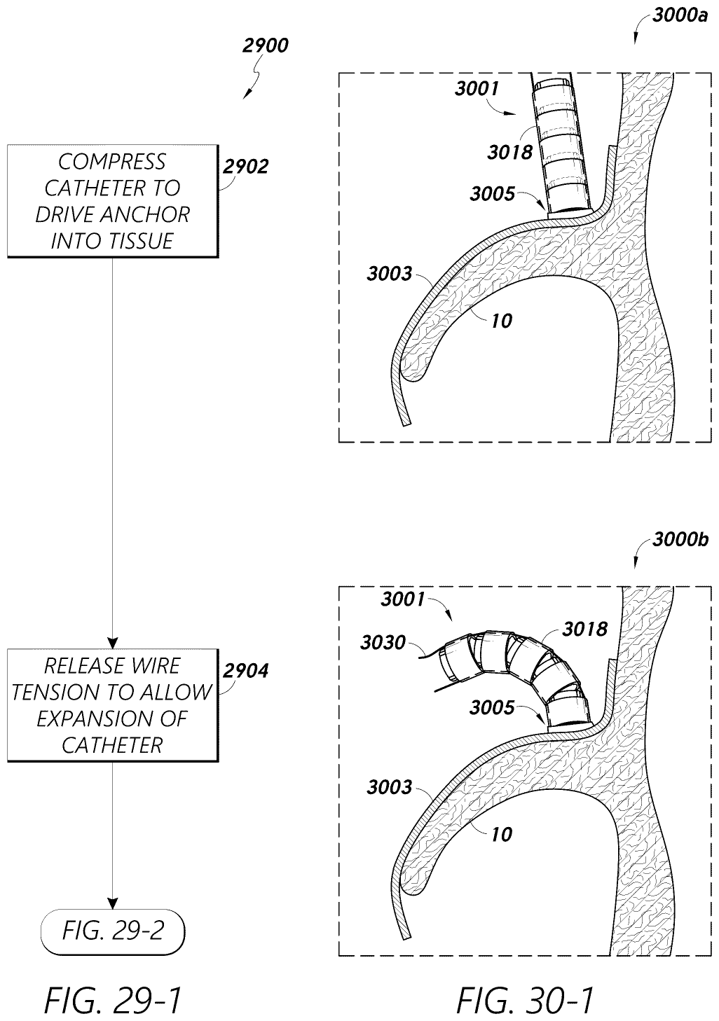

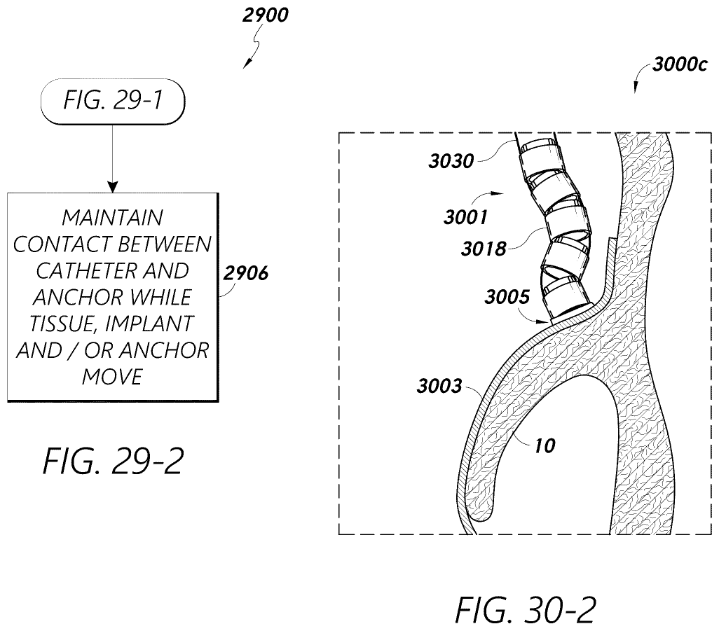

FIGS. 28A-28D illustrate an example flex portion 2800 of a delivery catheter in accordance with one or more implementations. The delivery catheter can be an anchor delivery system (which can be similar to other anchor delivery systems herein) or a portion/component of an anchor delivery system. In some implementations, the flex portion 2800 can comprise one or more pull wires 2830 configured to control positioning of one or more segments 2818 (e.g., rings) of the flex portion 2800. In some implementations, a delivery assembly can comprise the flex portion 2800 and/or a shaft (e.g., catheter) having a generally tubular form and/or comprising an inner lumen. The inner lumen can be configured to receive and/or facilitate conveyance of one or more wires and/or tissue anchors. The flex portion 2800 can similarly have a generally tubular form and/or can comprise an inner lumen. In some implementations, the flex portion 2800 can comprise a network of one or more segments 2818 interconnected by the one or more pull wires 2830. In some implementations, the segments 2818 can have a circular and/or cylindrical form.

In some implementations, one or more segments 2818 can comprise protrusions 2828 configured to mate with corresponding notches 2829 and/or apertures of another segment 2818. Accordingly, the one or more segments 2818 can be configured to join together and/or interconnect.

In some implementations, the flex portion 2800 can be configured to selectively move between multiple states, including an open state (e.g., an expanded and/or generally flexible state) illustrated in FIG. 28A, a closed state (e.g., a compressed and/or generally rigid state) illustrated in FIG. 28B, a bending state illustrated in FIG. 28C, and/or a zig-zag state illustrated in FIG. 28D. In the expanded state, there can be at least partial separation between the segments 2818 of the flex portion 2800. The one or more pull wires 2830 can be configured to interconnect the segments 2818 in the expanded state. Following delivery through narrow anatomical pathways and/or upon arriving at a target tissue site, the segments 2818 can be brought together by any suitable means to move the flex portion 2800 to the compressed state.

In some implementations, transitions between the various states of the flex portion 2800 can be controlled via the one or more pull wires 2830. For example, the pull wires 2830 can be selectively tensioned and/or pulled to press and/or pull, for example, a distal segment 2818a towards a proximal segment 2818b and/or to move the various segments 2818 together. To move all of the segments 2818 together, multiple pull wires 2830 can be tensioned and/or pulled simultaneously and/or in a synchronized manner such that all of the pull wires 2830 apply force to the segments 2818 to cause movement of the segments 2818 towards each other. As a result, the flex portion 2800 can be moved to the compressed form shown in FIG. 28B.

In some implementations, only a portion of the pull wires 2830 can be tensioned and/or pulled. For example, a first pull wire 2830a may not be tensioned and/or can have relatively low tension and/or a second pull wire 2830b can be tensioned and/or can have relatively high tension. As a result, the second pull wire 2830 may cause joining of the segments 2818 at one side of the flex portion 2800 and/or the first pull wire 2830a can allow for spacing between the segments 2818 at another side of the flex portion 2800. The flex portion 2800 can form a bend towards the second pull wire 2830b, as shown in FIG. 28C.

Relaxing and/or absence of tension in the one or more pull wires 2830 can allow for the flex portion 2800 to bend and/or migrate in various directions. For example, the flex portion 2800 can form a zig-zag shape in which the distal segment 2818a and the proximal segment 2818b can face different directions, as shown in FIG. 28D. The flex portion 2800 can be configured to move in response to movement of anatomy in contact with the flex portion 2800 to allow the flex portion 2800 to maintain contact with the anatomy and/or one or more anchors anchored into the anatomy while the anatomy moves.

In some implementations, the one or more pull wires 2830 can be configured to extend at least partially through the one or more segments 2818 to control positions of the one or more segments 2818 with respect to each other. For example, the one or more pull wires 2830 can pass through apertures of the segments 2818 and/or can be pulled to pull a distal segment 2818 towards the proximal segments 2818. By pulling the segments 2818 together, the one or more pull wires 2830 can be configured to facilitate movement of the flex portion 2800 from a flexible and/or expanded state to a compressed and/or rigid state.

The flex portion 2800 is shown comprising a first pull wire 2830a, a second pull wire 2830b, and/or additional pull wires 2830. However, the flex portion 2800 and/or delivery device can comprise any number of pull wires 2830. For example, the flex portion 2800 and/or delivery device can comprise four pull wires 2830. In some implementations, two pull wires 2830 can be situated at approximately opposing sides of a segment 2818, as shown in FIG. 28. In some implementations, the one or more pull wires 2830 can be configured to extend through each segment 2818 of the flex portion 2800. However, the one or more pull wires 2830 can be configured to extend through only a portion of the flex portion 2800.

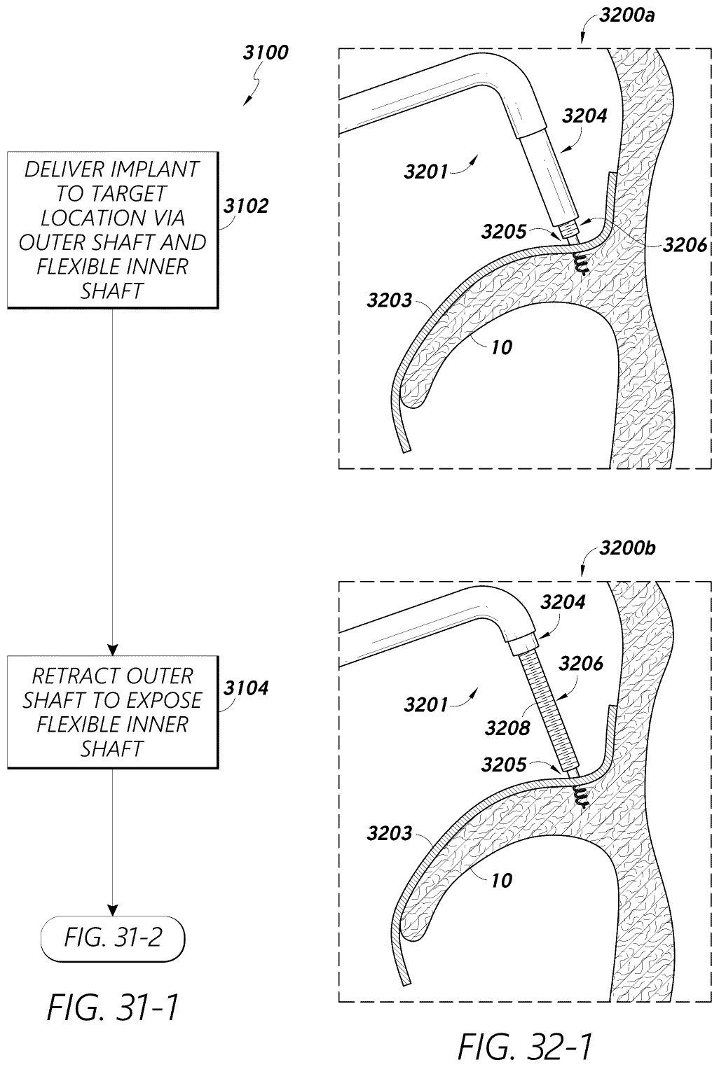

FIG. 29 (FIGS. 29-1 and 29-2) provides a flowchart illustrating an example method or process 2900 for driving one or more tissue anchors and/or for assessing anchoring of one or more tissue anchors in accordance with one or more examples. FIG. 30 (FIGS. 30-1 and 30-2) provides images associated with steps of the process 2900 of FIG. 29.

In some implementations, at step 2902, the method/process 2900 involves compressing a catheter 3001 (e.g., elongate shaft) and/or other delivery device and/or flex portion of a catheter 3001 to facilitate driving one or more anchors 3005 into tissue via the catheter 3001, as shown in image 3000a of FIG. 30. The catheter 3001 can be configured as an anchor delivery system (which can be similar to other anchor delivery systems herein) or can be a portion/component of an anchor delivery system. The catheter 3001 can be configured to be movable between multiple states and/or can comprise multiple forms. For example, during delivery through the body, the catheter 3001 can be configured to assume a generally flexible form (see, e.g., FIG. 28A and/or FIG. 28D). The catheter 3001 can comprise one or more pull wires 3030 configured to control positioning of various segments 3018 of the catheter 3001 relative to each other. During delivery, the one or more pull wires 3030 can have a generally relaxed state and/or can be configured to allow the segments 3018 to move independently of other segments 3018 and/or to allow gaps to form between the segments 3018. The catheter 3001 can comprise any number of segments 3018 and/or can comprise two or more segments 3018.

Prior to compressing the catheter 3001, the catheter 3001 and/or other delivery systems can be delivered via one or more blood vessels and/or heart chambers to a target tissue location. A flex portion of the catheter 3001 can be in a flexible state during delivery and/or can be moved to a compressed state using one or more pull wires 3030 and/or similar devices to facilitate driving one or more tissue anchors into the target tissue location. Pull wires 3030 can be configured to interconnect segments 3018 of the catheter 3001. The pull wires 3030 can be released to move the flex portion of the catheter 3001 from the compressed state to the flexible state. The catheter 3001 can be configured to carry one or more tissue anchors and/or to engage one or more tissue anchors (e.g., at a distal end of the catheter 3001).

Upon arrival at a target tissue site, the one or more pull wires 3030 can be tensioned and/or pulled to pull the segments 3018 together and/or to move the catheter 3001 to a generally compressed and/or rigid form. The rigid form of the catheter 3001 can allow the catheter 3001 to resist bending and/or twisting while the anchor 3005 is driven into the tissue (e.g., into a valve leaflet 10). In some implementations, the anchor 3005 can be configured to anchor a prosthetic leaflet 3003 and/or similar device.

The catheter 3001 can be configured to not bow and/or bend while in the compressed form shown in image 3000a. For example, driving pressure can be applied to the catheter 3001 without the catheter 3001 and/or the flex portion of the catheter 3001 bending.

In some implementations, at step 2904, the method/process 2900 involves releasing wire tension of one or more pull wires 3030 of the catheter 3001 to allow at least partial expansion of the catheter 3001, as shown in image 3000b of FIG. 30. For example, the catheter 3001 and/or flex portion of the catheter 3001 can bend in response to release of wire tension.

In some implementations, at step 2906, the method/process 2900 involves maintaining contact between the catheter 3001 and/or flex portion and/or distal end of the catheter 3001 and the anchor 3005 to assess a hold and/or anchoring of the anchor 3005 with the native tissue and/or leaflet 3003, as illustrated in image 3000c of FIG. 30. For example, the catheter 3001 and/or distal end of the catheter 3001 can be configured to move with the anchor 3005, prosthetic leaflet 3003, and/or tissue 10 while maintaining contact between the catheter 3001 and the anchor 3005.

If it is determined that the anchoring of the anchor 3005 is incomplete, the catheter 3001 and/or flex portion of the catheter 3001 can advantageously be compressed and/or tensioned to allow further driving of the anchor 3005 without requiring reattachment of the catheter 3001 to the anchor 3005. For example, the one or more pull wires 3030 can be in a flexible and/or untensioned state to allow movement of the catheter 3001 and/or can be tensioned to restore the compressed and/or rigid form of the catheter 3001. In the flexible state, the flex portion of the catheter 3001 can be configured to freely wobble to a rhythm of external forces and/or in various directions (e.g., complex three-dimensional and/or annular movement).

If the anchoring of the anchor 3005 is determined to be sufficient, the catheter 3001 and/or flex portion of the catheter 3001 can be removed from the anchor 3005 and/or can be moved to a different location to facilitate driving a second anchor into the prosthetic leaflet 3003, the tissue 10 and/or other target location.

The catheter 3001 can be configured to selectively assume stiff and/or soft states to assist with various stages of delivery and/or anchor driving. The catheter 3001 can be configured for transcatheter procedures. In some implementations, the catheter 3001 can be configured to facilitate navigation to a target location by assuming a flexible state and/or facilitate driving by assuming a stiff state. Moreover, the catheter 3001 can be configured reassume a flexible and/or soft state following driving of one or more anchors to continue driving one or more anchors following an assessment of one or more factors (e.g., mitral regurgitation) in response to initial driving of the one or more anchors.

It may be advantageous to acutely assess placement and/or anchoring of anchors and/or implants following delivery. In some implementations, the systems, apparatuses, devices, methods, etc. herein advantageously allow for acute assessment of one or more anchors and/or implants prior to removal of delivery systems. In some implementations, these advantageously allow for adjustment to delivered anchors and/or implants following and/or immediately following delivery and/or driving of the anchors and/or implants.

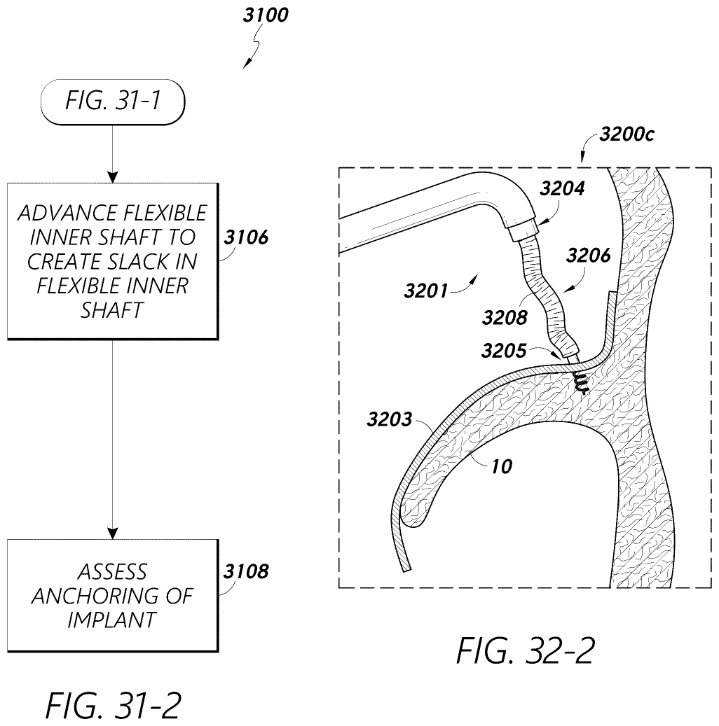

FIG. 31 (FIGS. 31-1 and 31-2) provides a flowchart illustrating an example method or process 3100 for driving one or more tissue anchors and/or for assessing anchoring of one or more tissue anchors in accordance with one or more examples. FIG. 32 (FIGS. 30-1 and 30-2) provides images associated with example steps of the process 3100 of FIG. 31.

At step 3102, the example method or process 3100 can involve delivering, driving, and/or anchoring one or more anchors including a first anchor 3205 at a target tissue site, as shown in image 3200a of FIG. 32. The first anchor 3205 can be delivered via a delivery system comprising a catheter 3201 (e.g., elongate shaft), an outer shaft 3204, and/or an inner shaft 3206. In some implementations, the catheter 3201 can be a steerable catheter 3201 and/or can be configured to bend and/or otherwise navigate through anatomical pathways. The catheter 3201 can comprise one or more pull wires configured to facilitate advancement and/or retraction of the outer shaft 3204 and/or inner shaft 3206.

In some implementations, the first anchor 3205 can be directed and/or placed using the outer shaft 3204 and/or the inner shaft 3206. In some implementations, the outer shaft 3204 can be at least partially rigid. For example, the outer shaft 3204 can be at least partially resistant to bending. The outer shaft 3204 can be configured to provide sufficient rigidity and/or stiffness to allow the outer shaft 3204 to be extended to cause driving and/or anchoring of the first anchor 3205. The outer shaft 3204 can comprise sufficient push-ability and/or bend rigidity for driving through an implant 3203 and/or native tissue. In some implementations, the outer shaft 3204 can comprise a braided polymer and/or laser-cut hypotube.

In some implementations, the inner shaft 3206 can be at least partially sheathed within the outer shaft 3204 and/or the outer shaft 3204 can be at least partially sheathed within the catheter 3201 during delivery. For example, the outer shaft 3204 can comprise an inner lumen sized to fit at least partially around the inner shaft 3206 and/or the inner shaft 3206 can be sized and/or shaped to fit within the outer shaft 3204.

In some implementations, the outer shaft 3204 can be extended beyond a distal end of the catheter 3201 during direction and/or placement of the first anchor 3205 to allow the stiffness of the outer shaft 3204 to facilitate driving of the first anchor 3205.

In some implementations, the inner shaft 3206 can be at least partially flexible. In some implementations, the inner shaft 3206 can be extended with the outer shaft 3204 during and/or driving and/or placement of the first anchor 3205. However, the outer shaft 3204 can be configured to at least partially prevent bending and/or movement of the inner shaft 3206 during driving and/or delivery of the first anchor 3205.

In some implementations, the outer shaft 3204 and/or catheter 3201 may not be directly coupled to the first anchor 3205. For example, the inner shaft 3206 can be configured to couple to the first anchor 3205 at a distal end of the inner shaft 3206. Accordingly, extension of the inner shaft 3206 may cause corresponding extension of the first anchor 3205 towards the implant 3203. The inner shaft 3206 and outer shaft 3204 can be extended together and/or separately. For example, extension of the outer shaft 3204 can be configured to cause corresponding extension of the inner shaft 3206 and/or extension of the inner shaft 3206 can be configured to cause corresponding extension of the outer shaft 3204. However, in some implementations, the outer shaft 3204 and inner shaft 3206 can be extended separately and/or via separate driving components.

At step 3104, the process 3100 can involve at least partially retracting the outer shaft 3204 and/or at least partially exposing a distal portion of the inner shaft 3206, as shown in image 3200b of FIG. 32. Retracting the outer shaft 3204 can involve pulling the outer shaft 3204 further into the catheter 3201 and/or away from the first anchor 3205.

In some implementations, the outer shaft 3204 can be retracted once the first anchor 3205 is at least partially driven, anchored, placed, and/or fixtured to the implant 3203 and/or anatomy below the implant 3203. Retraction of the outer shaft 3204 may result in exposure of the inner shaft 3206.

In some implementations, the inner shaft 3206 can have a generally flexible structure and/or can be configured to bend in response to pressure against the inner shaft 3206. In some implementations, the inner shaft 3206 can comprise a laser-cut hypotube and/or can comprise one or more features configured to enhance flexibility of the inner shaft 3206. For example, the inner shaft 3206 can have a generally thin structure and/or can be thinner relative to the outer shaft 3204. In some implementations, the inner shaft 3206 can comprise one or more cavities 3208 (e.g., openings, slits, apertures, holes, grooves, etc.) configured to improve flexibility of the inner shaft 3206. The one or more cavities 3208 can extend entirely and/or partially through walls of the inner shaft 3206. For examples, the one or more cavities 3208 can provide openings to an inner lumen of the inner shaft 3206. However, the one or more cavities can comprise grooves cut partially out of the walls of the inner shaft 3206.

In some implementations, the inner shaft 3206 can be configured to bend in multiple directions. For example, the inner shaft 3206 can be configured to bend to a generally wavy and/or oscillating form. Accordingly, the inner shaft 3206 can comprise cavities 3208 on multiple sides and/or along a substantial length of the inner shaft 3206 (e.g., along the entire inner shaft 3206).

In some implementations, the inner shaft 3206 can comprise cavities 3208 on one or more sides of the inner shaft 3206 and/or may not comprise cavities 3208 on one or more sides of the inner shaft 3206.

In some implementations, the inner shaft 3206 can comprise cavities 3208 only at a distal end of the inner shaft 3206 and/or not along an entire length of the inner shaft 3206.

The inner shaft 3206 is illustrated as a tube with a solid circumferential wall, however the inner shaft 3206 can have different forms. For example, the inner shaft 3206 can comprise a coil and/or a coiled wire and/or line. In some implementations, the inner shaft 3206 can comprise an inflatable tube and/or balloon configured to be pressurized and/or depressurized with one or more gases and/or fluids.

In some implementations, the inner shaft 3206 can be configured to maintain contact with the first anchor 3205 following retraction of the outer shaft 3204. For example, the inner shaft 3206 can be coupled to the first anchor 3205 and/or can be configured to grasp the first anchor 3205 and/or to maintain the grasp on the first anchor 3205 following retraction of the outer shaft 3204.

At step 3106, the method/process 3100 can involve at least partially advancing the inner shaft 3206 further beyond the outer shaft 3204 and/or catheter 3201 to create slack and/or promote bending of the inner shaft 3206, as shown in image 3200c of FIG. 32. As the inner shaft 3206 is advanced, the catheter 3201 and/or outer shaft 3204 can remain in place. Accordingly, a distance between the first anchor 3205 and the outer shaft 3204 can remain constant. As the inner shaft 3206 is advanced, a greater amount of the inner shaft 3206 can be extended and/or can fill the space between the outer shaft 3204 and the first anchor 3205.

In some implementations, the inner shaft 3206 can be configured to naturally bend to a bent and/or wavy form to allow the distal portion of the inner shaft 3206 to fill the smaller distance and/or space. In some implementations, the inner shaft 3206 and/or outer shaft 3204 can be configured to be advanced and/or retracted independently of each other. For example, advancement and/or retraction of outer shaft 3204 may not cause corresponding advancement and/or retraction of inner shaft 3206.

In some implementations, advancing the inner shaft 3206 can be configured to reduce a load from the inner shaft 3206 on the first anchor 3205 and/or to create slack in the inner shaft 3206. For example, bending and/or slack in the inner shaft 3206 may reduce a pulling force from the distal end of the inner shaft 3206 on the first anchor 3205. In the advanced state, the distal end of the inner shaft 3206 can be configured to loosely grasp the first anchor 3205 and/or allow a maximum amount of natural movement of the tissue anchor 3205 and/or implant 3203 in response to movement of the tissue.

In some implementations, the inner shaft 3206 can be at least partially inflatable and/or compressible. For example, the inner shaft 3206 can be configured to be inflated with a gas and/or fluid during delivery to facilitate grasping and/or driving of the first anchor 3205. The gas and/or fluid can be suctioned out of the inner shaft 3206 and/or the inner shaft 3206 can be depressurized before or following retraction of the outer shaft 3204 to soften and/or increase flexibility of the inner shaft 3206.

In the flexible state, the inner shaft 3206 can be configured to freely wobble and/or bend to a rhythm of external forces and/or in various (e.g., multiple) directions (e.g., complex three-dimensional and/or annular movement).

At step 3108, the method/process 3100 can involve assessing the anchoring and/or placement of the implant 3203 and/or first anchor 3205 with the inner shaft 3206 in a slackened and/or loose form. The slackened inner shaft 3206 can allow movement of the anchor 3205 and/or implant 3203. Accordingly, the first anchor 3205 and/or implant 3203 can be viewed while the inner shaft 3206 is in the slackened state to determine if the first anchor 3205 and/or implant 3203 move naturally and/or in a desired manner.

Any suitable means of imaging can be used to assess the placement and/or anchoring of the first anchor 3205 and/or additional anchors and/or implant 3203. For example, echo and/or fluoro imaging can be utilized in assessment of the anchor(s) and/or implant 3203. While the process 3100 described placement of only a first anchor 3205 additional anchors can be delivered, assessed, and/or anchored in a similar manner.

If it is determined that the anchoring of the anchor 3205 is incomplete, the inner shaft 3206 can advantageously be at least partially retracted (or pressurized) and/or the outer shaft 3204 can advantageously be advanced to allow further driving of the anchor 3205 without requiring reattachment of the inner shaft 3206 to the anchor 3205.

If the anchoring of the anchor 3205 is determined to be sufficient, the catheter 3201, outer shaft 3204 and/or inner shaft 3206 can be removed from the anchor 3205 and/or can be moved to a different location to facilitate driving a second anchor into the prosthetic leaflet 3203, the tissue 10 and/or other target location.

In some implementations, the outer shaft 3204 can be advanced to continue driving one or more anchors following an assessment of one or more factors (e.g., mitral regurgitation) in response to initial driving of the one or more anchors.

In some implementations, multiple anchors can be implanted and/or assessed simultaneously.

In some implementations, a system for assessing anchoring of one or more tissue anchors includes: an outer shaft including an inner lumen; and an inner shaft configured to fit at least partially within the inner lumen of the outer shaft and drive one or more tissue anchors, wherein the inner shaft has a generally flexible structure relative to the outer shaft.

In some implementations, the techniques described herein relate to a system, wherein the inner shaft includes one or more cavities configured to improve flexibility of the inner shaft.

In some implementations, the techniques described herein relate to a system, wherein the outer shaft is configured to advance and retract independently of the inner shaft.

In accordance with some implementations of the present disclosure, a method for assessing anchoring of one or more tissue anchors includes delivering an outer shaft and an inner shaft to a target tissue location, the outer shaft at least partially enclosing the inner shaft.

In some implementations, the method further includes attaching the inner shaft to a tissue anchor.

In some implementations, the method further includes retracting the outer shaft to expose a distal portion of the inner shaft.

In some implementations, the method further includes advancing the inner shaft to create slack in the distal portion of the inner shaft.

In some implementations, the method further includes assessing the tissue anchor with the inner shaft attached to the tissue anchor.

In some implementations, the method further includes advancing the outer shaft to drive the tissue anchor into tissue.

In some implementations, the inner shaft is generally flexible relative to the outer shaft. In some implementations, the inner shaft includes one or more cavities configured to improve flexibility of the inner shaft.

In some implementations, the outer shaft is configured to advance and retract independently of the inner shaft.

In some implementations, the method further includes, in response to determining that the tissue anchor is suitably anchored, removing the inner shaft from the tissue anchor.

In some implementations, advancing the inner shaft to create slack in the distal portion of the inner shaft involves allowing the distal portion of the inner shaft to bend in multiple directions.

In some implementations, the method further includes detaching the inner shaft from the tissue anchor.

In some implementations, the method further includes attaching the inner shaft to a second tissue anchor. In some implementations, the method further includes retracting the outer shaft to expose a distal portion of the inner shaft.

In some implementations, the method further includes advancing the inner shaft to create slack in the distal portion of the inner shaft.

In some implementations, the method further includes assessing the second tissue anchor with the inner shaft attached to the second tissue anchor.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In accordance with some implementations of the present disclosure, a method for delivering one or more tissue anchors comprises delivering an elongate shaft carrying one or more tissue anchors at a distal end of the elongate shaft to a target tissue location.

In some implementations, the elongate shaft comprises a flex portion configured to move between a first state and a second state.

In some implementations, the method can further comprise moving the flex portion from the first state to the second state to facilitate driving the one or more tissue anchors into the target tissue location and moving the flex portion from the second state to the first state.

In some implementations, moving the flex portion from the first state to the second state can involve compressing the flex portion.

In some implementations, moving the flex portion from the first state to the second state involves twisting the elongate shaft until a gap forms in the flex portion.

In some implementations, the flex portion comprises at least one detachable tab between segments of the flex portion. The segments can be ring-shaped.

In some implementations, the flex portion can comprise a network of interlocking teeth. In some implementations, the flex portion comprises a coil extending through a lumen of the flex portion.

In some implementations, the coil is configured to interconnect segments of the flex portion. In some implementations, the flex portion can comprise a cut through the elongate shaft.

In some implementations, moving the flex portion from the first state to the second state involves pulling one or more pull wires extending at least partially through the flex portion.

In some implementations, the flex portion includes two or more segments. In some implementations, the one or more pull wires interconnect the two or more segments.

In some implementations, moving the flex portion from the second state to the first state involves releasing the one or more pull wires.

In some implementations, in response to determining that the first tissue anchor is not suitably anchored, the method includes moving the flex portion from the first state to the second state to facilitate further driving the first tissue anchor into the target tissue location.

In some implementations, in response to determining that the first tissue anchor is suitably anchored, the method includes removing the flex portion from the first tissue anchor.

In some implementations, the method further comprises moving the first tissue anchor from the first state to the second state to facilitate driving a second tissue anchor into a second target tissue location.

Any of the above method(s) can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can comprise, for example, computerized and/or physical representations.

In some implementations, a system for delivering one or more tissue anchors comprises an elongate shaft carrying one or more tissue anchors at a distal end of the elongate shaft to a target tissue location. In some implementations, the elongate shaft comprises a flex portion configured to move between a first state and a second state.

In some implementations, the system further comprises an inner shaft configured to extend at least partially through a lumen of the elongate shaft. In some implementations, the system can further comprise an outer shaft configured to at least partially enclose the elongate shaft.

In some implementations, the flex portion can be configured to move from the first state to the second state by compressing the flex portion.

In some implementations, the flex portion is configured to form a gap in response to twisting of the elongate shaft.

In some implementations, the flex portion comprises at least one detachable tab between segments of the flex portion. The segments can be ring-shaped.

In some implementations, the flex portion can comprise a network of interlocking teeth. In some implementations, the flex portion comprises a coil extending through a lumen of the flex portion.

In some implementations, the coil is configured to interconnect segments of the flex portion. The flex portion can comprise a cut through the elongate shaft.

In some implementations, the system can further comprise one or more pull wires configured to extend at least partially through the flex portion. In some implementations, the one or more pull wires extend at least partially through multiple segments of the flex portion.

Any of the various systems, assemblies, devices, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, apparatus, etc. (e.g., with heat, radiation, ethylene oxide, hydrogen peroxide, etc.).

### ADDITIONAL DESCRIPTION OF EXAMPLES

Provided below is a list of examples, each of which can include aspects of any of the other examples disclosed herein. Furthermore, aspects of any example described above can be implemented in any of the numbered examples provided below.

Depending on the example, certain acts, events, or functions of any of the processes or algorithms described herein can be performed in a different sequence, can be added, merged, or left out altogether. Thus, in certain examples, not all described acts or events are necessary for the practice of the processes.

Some implementations of the present disclosure relate to various examples, including the following.

Example 1: A system for assessing anchoring of one or more tissue anchors, the system comprising: an outer shaft comprising an inner lumen; and an inner shaft configured to fit at least partially within the inner lumen of the outer shaft and drive one or more tissue anchors, wherein the inner shaft has a generally flexible structure relative to the outer shaft.

Example 2: The system of any example herein, in particular example 1, wherein the inner shaft comprises one or more cavities configured to improve flexibility of the inner shaft.

Example 3: The system of any example herein, in particular examples 1-2, wherein the outer shaft is configured to advance and retract independently of the inner shaft.

Example 4: A method for assessing anchoring of one or more tissue anchors, the method comprising delivering an outer shaft and an inner shaft to a target tissue location, the outer shaft at least partially enclosing the inner shaft; attaching the inner shaft to a tissue anchor; retracting the outer shaft to expose a distal portion of the inner shaft; advancing the inner shaft to create slack in the distal portion of the inner shaft; and assessing the tissue anchor with the inner shaft attached to the tissue anchor.

Example 5: The method of any example herein, in particular example 4, further comprising advancing the outer shaft to drive the tissue anchor into tissue.

Example 6: The method of any example herein, in particular examples 4-5, wherein the inner shaft is generally flexible relative to the outer shaft.

Example 7: The method of any example herein, in particular example 6, wherein the inner shaft comprises one or more cavities configured to improve flexibility of the inner shaft.

Example 8: The method of any example herein, in particular examples 4-7, wherein the outer shaft is configured to advance and retract independently of the inner shaft.

Example 9: The method of any example herein, in particular examples 4-8, further comprising, in response to determining that the tissue anchor is suitably anchored, removing the inner shaft from the tissue anchor.

Example 10: The method of any example herein, in particular examples 4-9, wherein the inner shaft comprises one or more cavities configured to improve flexibility of the inner shaft.

Example 11: The method of any example herein, in particular examples 4-10, wherein the outer shaft is configured to advance and retract independently of the inner shaft.

Example 12: The method of any example herein, in particular examples 4-11, wherein advancing the inner shaft to create slack in the distal portion of the inner shaft involves allowing the distal portion of the inner shaft to bend in multiple directions.

Example 13: The method of any example herein, in particular examples 4-12, further comprising: detaching the inner shaft from the tissue anchor; attaching the inner shaft to a second tissue anchor; retracting the outer shaft to expose a distal portion of the inner shaft; advancing the inner shaft to create slack in the distal portion of the inner shaft; and assessing the second tissue anchor with the inner shaft attached to the second tissue anchor.

Example 14: A method for delivering one or more tissue anchors, the method comprising: delivering an elongate shaft to a target tissue location, the elongate shaft comprising a flex portion configured to move between a first state and a second state; moving the flex portion from the first state to the second state to facilitate driving a first tissue anchor into the target tissue location; and moving the flex portion from the second state to the first state while maintaining contact between the flex portion and the first tissue anchor.

Example 15: The method of any example herein, in particular example 14, wherein the elongate shaft is configured to carry one or more tissue anchors at a distal end of the elongate shaft.

Example 16: The method of any example herein, in particular examples 14-15, wherein moving the flex portion from the first state to the second state involves compressing the flex portion.

Example 17: The method of any example herein, in particular examples 14-16, wherein the flex portion comprises two or more segments.

Example 18: The method of any example herein, in particular example 17, wherein the segments are ring-shaped.

Example 19: The method of any example herein, in particular example 17, wherein moving the flex portion from the first state to the second state involves pulling one or more pull wires extending at least partially through the two or more segments.

Example 20: The method of any example herein, in particular example 19, wherein the one or more pull wires interconnect the two or more segments.

Example 21: The method of any example herein, in particular example 19, wherein moving the flex portion from the second state to the first state involves releasing the one or more pull wires.

Example 22: The method of any example herein, in particular examples 14-21, further comprising, in response to determining that the first tissue anchor is not suitably anchored, moving the flex portion from the first state to the second state to facilitate further driving the first tissue anchor into the target tissue location.

Example 23: The method of any example herein, in particular examples 14-22, further comprising, in response to determining that the first tissue anchor is suitably anchored, removing the flex portion from the first tissue anchor.

Example 24: The method of any example herein, in particular examples 23, further comprising moving the first tissue anchor from the first state to the second state to facilitate driving a second tissue anchor into a second target tissue location.

Example 25: A system for delivering one or more tissue anchors, the system comprising: a handle; a threaded screw coupled to the handle; a knob; an outer shaft coupled to the knob; and a bolt configured to twist along the threaded screw and press against the knob.

Example 26: The system of any example herein, in particular example 25, further comprising an inner shaft configured to extend at least partially through the knob and the threaded screw.

Example 27: The system of any example herein, in particular example 26, further comprising a peg extending from the inner shaft.

Example 28: The system of any example herein, in particular example 27, wherein the knob comprises a channel configured to receive the peg.

Example 29: The system of any example herein, in particular example 28, wherein the peg is configured to prevent rotation of the knob.

Example 30: The system of any example herein, in particular examples 25-29, further comprising an arm coupled to the bolt, the arm configured to press against or pull a protrusion extending from an outer surface of the knob.

Example 31: The system of any example herein, in particular examples 25-30, further comprising a stopper nut disposed along the threaded screw and between the bolt and the handle.

Example 32: The system of any example herein, in particular examples 25-31, further comprising a washer disposed between the knob and the bolt.

Example 33: The system of any example herein, in particular examples 25-32, wherein the knob is configured to not twist in response to force from the bolt.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without author input or prompting, whether these features, elements and/or steps are included or are to be performed in any particular implementation. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. can be either X, Y or Z. Thus, such conjunctive language is not generally intended to imply that certain implementations require at least one of X, at least one of Y and at least one of Z to each be present.

It should be appreciated that in the above description of implementations, various features are sometimes grouped together in a single implementation, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular implementation herein can be applied to or used with any other implementation(s). Further, no component, feature, step, or group of components, features, or steps are necessary or indispensable for each implementation. Thus, it is intended that the scope of the inventions herein disclosed and claimed below should not be limited by the particular implementations described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example implementations belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, can be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device can be placed “above” another device. Accordingly, the illustrative term “below” can include both the lower and upper positions. The device can also be oriented in the other direction, and thus the spatially relative terms can be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method comprising:
delivering an elongate shaft to a target tissue location, the elongate shaft comprising a flex portion configured to move between a first state and a second state;
moving the flex portion from the first state to the second state to facilitate driving a first tissue anchor into the target tissue location; and
moving the flex portion from the second state to the first state while maintaining contact between the flex portion and the first tissue anchor.

2. The method of claim 1, wherein moving the flex portion from the first state to the second state involves compressing the flex portion.

3. The method of claim 1, wherein the flex portion comprises two or more ring-shaped segments.

4. The method of claim 3, wherein moving the flex portion from the first state to the second state involves pulling one or more pull wires extending at least partially through the two or more ring-shaped segments.

5. The method of claim 4, wherein moving the flex portion from the second state to the first state involves releasing the one or more pull wires.

6. The method of claim 1, further comprising, in response to determining that the first tissue anchor is not suitably anchored, moving the flex portion from the first state to the second state to facilitate further driving the first tissue anchor into the target tissue location.

7. The method of claim 1, further comprising, in response to determining that the first tissue anchor is suitably anchored, removing the flex portion from the first tissue anchor.

8. A system comprising:
an outer shaft comprising an inner lumen; and
an inner shaft configured to fit at least partially within the inner lumen of the outer shaft and drive one or more tissue anchors, wherein the inner shaft has a generally flexible structure relative to the outer shaft and comprises one or more cavities or gaps configured to improve flexibility of the inner shaft.

9. The system of claim 8, wherein the outer shaft is configured to advance and retract independently of the inner shaft.

10. The system of claim 8, wherein the inner shaft comprises two or more segments.

11. The system of claim 10, further comprising one or more pull wires extending at least partially through the two or more segments.

12. A method comprising:
delivering an outer shaft and an inner shaft to a target tissue location, the outer shaft at least partially enclosing the inner shaft;
attaching the inner shaft to a tissue anchor;
retracting the outer shaft to expose a distal portion of the inner shaft;
advancing the inner shaft to create slack in the distal portion of the inner shaft; and
assessing the tissue anchor with the inner shaft attached to the tissue anchor.

13. The method of claim 12, further comprising advancing the outer shaft to drive the tissue anchor into tissue.

14. The method of claim 13, wherein the inner shaft is more flexible relative to the outer shaft.

15. The method of claim 14, wherein the inner shaft comprises one or more cavities or gaps configured to improve flexibility of the inner shaft.

16. The method of claim 13, wherein the outer shaft is configured to advance and retract independently of the inner shaft.

17. The method of claim 12, further comprising, in response to determining that the tissue anchor is suitably anchored, removing the inner shaft from the tissue anchor.

18. The method of claim 12, wherein the inner shaft comprises two or more segments.

19. The method of claim 12, wherein advancing the inner shaft to create slack in the distal portion of the inner shaft involves allowing the distal portion of the inner shaft to bend in multiple directions.

20. The method of claim 12, further comprising:
detaching the inner shaft from the tissue anchor;
attaching the inner shaft to a second tissue anchor;
retracting the outer shaft to expose a distal portion of the inner shaft;
advancing the inner shaft to create slack in the distal portion of the inner shaft; and
assessing the second tissue anchor with the inner shaft attached to the second tissue anchor.
